(19) United States
(12) Patent Application Publication
(10) Pub. No.: US 2020/0031848 A1
(43) Pub. Date: Jan. 30, 2020

(54) AR-V7 INHIBITORS

(71) Applicants: Hoffmann-La Roche Inc., Little Falls, NJ (US); The University of British Columbia, Vancouver (CA)

(72) Inventors: Fuqiang BAN, Vancouver (CA); Kush DALAL, Vancouver (CA); Alexander FLOHR, Basel (CH); Eric J. J. LEBLANC, Vancouver (CA); Huifang LI, Vancouver (CA); Paul S. RENNIE, Vancouver (CA); Artem TCHERKASSOV, Vancouver (CA)

(21) Appl. No.: 16/469,237

(22) PCT Filed: Dec. 18, 2017
(86) PCT No.: PCT/EP2017/083281

§ 371 Date: Jun. 13, 2019

(30) Foreign Application Priority Data
Dec. 19, 2016 (EP) 16205150.2

(51) Int. Cl. C07D 498/08; C07D 417/14; C07D 498/10

(52) U.S. Cl. CPC C07D 498/08; C07D 498/10; C07D 417/14

(57) ABSTRACT

The present invention provides compounds having Ar-V7 inhibitory activity, their manufacture, pharmaceutical compositions containing them and their use as therapeutically active substances. The active compounds of the present invention are useful in the therapeutic and/or prophylactic treatment of cancer.

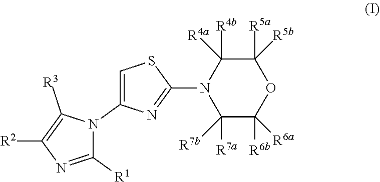

### BACKGROUND ART

The Androgen receptor (AR) is a nuclear receptor. AR-V7 is a hormone-independent splice variant of the androgen receptor. Inhibition of AR-V7 has been associated with various indications, in particular cancers such as prostate cancer (Krause et al.¹ and Scher et al.²) Prostate cancer is the second most frequently diagnosed cancer, and the third most common cause of death from cancer in men in developed countries (Damber et al.³). The importance of AR signaling is also recognized in breast cancer (Hickey et al.⁴) and in various other non-prostatic malignancies (Antonarakis⁵).

Luo describe that AR-V7 can be measured in tumor cells derived from cancer patients.⁶

Effective inhibition of human AR is thus regarded as effective therapeutic approach to the treatment of various cancers, in particular prostate cancer. There is a need for new and useful AR-V7 inhibitors in pharmaceutical science.

WO2015120543⁷ describes compounds as AR-V7 inhibitors and their use in the treatment of various indications such as prostate cancer. WO2015065919⁸ describes compositions of an AR-V7 inhibitor and their use in the treatment of various indications such as prostate cancer.

Li et al.⁹ describe small-molecule inhibitors selectively targeting the DNA-binding domain of the human androgen receptor. Dalal et al.¹⁰ describe compounds selectively targeting the DNA binding domain of the androgen receptor as a prospective therapy for prostate cancer.

The present invention provides novel compounds of formula I, their manufacture, medicaments based on a compound in accordance with the invention and their production as well as the use of compounds of formula I in the control or prevention of illnesses such as cancers, in particular prostate cancer, breast cancer, bladder cancer, endometrial and ovarian cancers, hepatocellular and pancreatic cancers, and salivary gland cancers. The novel compounds of formula I have good activity and pharmacological properties.

### FIELD OF THE INVENTION

The present invention provides bromoimidazolyl compounds having AR-V7 inhibitors properties, their manufacture, pharmaceutical compositions containing them and their use as therapeutically active substances.

### SUMMARY OF THE INVENTION

The present invention provides a compound of formula I,

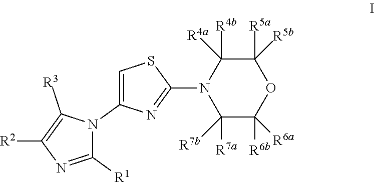

wherein the substituents and variables are as described below and in the claims, or a pharmaceutically acceptable salt thereof.

The present compounds have AR-V7 inhibitors properties and may therefore be used in the therapeutic and/or prophylactic treatment of cancers, in particular prostate cancer, breast cancer, bladder cancer, endometrial and ovarian cancers, hepatocellular and pancreatic cancers, and salivary gland cancers.

### DETAILED DESCRIPTION OF THE INVENTION

The present invention provides a compound of formula I and their pharmaceutically acceptable salts thereof, the preparation of the above mentioned compounds, medicaments containing them and their manufacture as well as the use of the above mentioned compounds in the therapeutic and/or prophylactic treatment of cancers, in particular prostate cancer, breast cancer, bladder cancer, endometrial and ovarian cancers, hepatocellular and pancreatic cancers, and salivary gland cancers.

The following definitions of the general terms used in the present description apply irrespectively of whether the terms in question appear alone or in combination with other groups.

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

The term “C₁₋₆-alkyl”, alone or in combination with other groups, stands for a hydrocarbon radical which may be linear or branched, with single or multiple branching, wherein the alkyl group in general comprises 1 to 6 carbon atoms, for example, methyl (Me), ethyl (Et), propyl, isopropyl (i-propyl), n-butyl, i-butyl (isobutyl), 2-butyl (sec-butyl), t-butyl (tert-butyl), isopentyl, 2-ethyl-propyl (2-methyl-propyl), 1,2-dimethyl-propyl and the like. Particular “C₁₋₆-alkyl” are “C₁₋₃-alkyl”. Specific groups are methyl and ethyl.

The term “halogen-C₁₋₆-alkyl”, alone or in combination with other groups, refers to C₁₋₆-alkyl as defined herein, which is substituted by one or multiple halogen, particularly 1-5 halogen, more particularly 1-3 halogen. Particular halogen is fluoro. Particular “halogen-C₁₋₆-alkyl” is fluoro-C₁₋₆-alkyl and a particular “halogen-C₁₋₃-alkyl” is fluoro-C₁₋₃-alkyl. Examples are trifluoromethyl, difluoromethyl, fluoromethyl and the like. A specific group is CF₃.

The term “hydroxy-C₁₋₆-alkyl”, alone or in combination with other groups, refers to C₁₋₆-alkyl as defined herein, which is substituted by one or multiple hydroxy, particularly 1 hydroxy.

A specific group is hydroxy-CH₂CH₃.

The term “cyano”, alone or in combination with other groups, refers to N≡C—(NC—).

The term “halogen”, alone or in combination with other groups, denotes chloro (Cl), iodo (I), fluoro (F) and bromo (Br). Particular “halogen” is Br, Cl and I. A specific group is Br.

The term “heteroaryl”, alone or in combination with other groups, refers to an aromatic carbocyclic group of having a single 4 to 8 membered ring, in particular 5 to 8, or multiple condensed rings comprising 6 to 14, in particular 6 to 10 ring atoms and containing 1, 2 or 3 heteroatoms individually selected from N, O and S, in particular 1N or 2N, in which group at least one heterocyclic ring is aromatic. Examples of “heteroaryl” include benzofuryl, benzoimidazolyl, 1H-benzoimidazolyl, benzooxazinyl, benzoxazolyl, benzothiazinyl, benzothiazolyl, benzothienyl, benzotriazolyl, furyl, imidazolyl, indazolyl, 1H-indazolyl, indolyl, isoquinolinyl, isothiazolyl, isoxazolyl, oxazolyl, pyrazinyl, pyrazolyl (pyrazyl), 1H-pyrazolyl, pyrazolo[1,5-a]pyridinyl, pyridazinyl, pyridinyl, pyrimidinyl, pyrrolyl, quinolinyl, tetrazolyl, thiazolyl, thienyl, triazolyl, 6,7-dihydro-5H-[1]pyrindinyl and the like. A specific group is thiazolyl.

The term “C₁₋₆-alkoxy”, alone or in combination with other groups, stands for an —O—C₁₋₆-alkyl radical which may be linear or branched, with single or multiple branching, wherein the alkyl group in general comprises 1 to 6 carbon atoms, for example, methoxy (OMe, MeO), ethoxy (OEt), propoxy, isopropoxy (i-propoxy), n-butoxy, i-butoxy (iso-butoxy), 2-butoxy (sec-butoxy), t-butoxy (tert-butoxy), isopentyloxy (i-pentyloxy) and the like. Particular “C₁₋₆-alkoxy” are groups with 1 to 4 carbon atoms. Specific is methoxy.

The term “aryl” denotes a monovalent aromatic carbocyclic mono- or bicyclic ring system comprising 6 to 10 carbon ring atoms. Examples of aryl moieties include phenyl and benzyl.

The term “C₃₋₇-cycloalkyl”, alone or in combination with other groups, denotes a monovalent saturated monocyclic or bicyclic hydrocarbon group of 3 to 7 ring carbon atoms, particularly a monovalent saturated monocyclic hydrocarbon group of 3 to 5 ring carbon atoms. Bicyclic means consisting of two saturated carbocycles having one or more carbon atoms in common. Particular cycloalkyl groups are monocyclic. Examples for monocyclic cycloalkyl are cyclopropyl, cyclobutanyl, cyclopentyl, cyclohexyl or cycloheptyl. A specific group is cyclopropyl.

The term “cycloheteroalkyl”, alone or in combination with other groups, denotes a monovalent saturated or partly unsaturated ring system of 4 to 11 ring atoms, comprising 1, 2, or 3 ring heteroatoms selected from N, O and S, the remaining ring atoms being carbon. Bicyclic means consisting of two cycles having two ring atoms in common, i.e. the bridge separating the two rings is either a single bond or a chain of one or two ring atoms. Examples for monocyclic saturated heterocycloalkyl are 1,1-dioxo-thiomorpholin-4-yl, azepanyl, azetidinyl, diazepanyl, homopiperazinyl, imidazolidinyl, isoxazolidinyl, morpholinyl, oxanyl, oxazepanyl, oxazolidinyl, piperazinyl, piperidinyl, pyrazolidinyl, pyrrolidinyl, tetrahydrofuranyl, tetrahydropyranyl, tetrahydro-thienyl, tetrahydrothiopyranyl, thiazolidinyl, thiomorpholinyl and the like. Examples for bicyclic saturated heterocycloalkyl are 1,9-dioxa-4-azaspiro[5.5]undecanyl, 2,3,4a,5,6,7,8,8a-octahydrobenzo[b][1,4]oxazinyl, 2-azaspiro[3.5]nonan-2-yl, 2-oxa-5-azabicyclo[2.2.1]heptanyl, 2-oxa-6-azaspiro[3.3]heptanyl, 3,4a,5,6,7,7a-hexahydro-2H-cyclopenta[b][1,4]oxazinyl or 2-azaspiro[3.3]heptan-2-yl, 3-oxa-8-azabicyclo[3.2.1]octanyl, 3-oxa-9-aza-bicyclo[3.3.1]nonyl, 3-thia-9-aza-bicyclo[3.3.1]nonyl, 6-oxa-9-azaspiro[4.5]decanyl, 7-oxa-2-azaspiro[3.5]nonanyl, 8-aza-bicyclo[3.2.1]octyl, 8-oxa-3-aza-bicyclo[3.2.1]octyl, 9-aza-bicyclo[3.3.1]nonyl, quinuclidinyl, and the like. Examples for partly unsaturated heterocycloalkyl are dihydrofuryl, dihydro-oxazolyl, dihydropyranyl, imidazolinyl, tetrahydro-pyridinyl, and the like. Specific groups are 1,4-oxepanyl, 1,4-thiazinanyl 1,1-dioxide, 1,9-dioxa-4-azaspiro[5.5]undecanyl, 2,3,4a,5,6,7,8,8a-octahydrobenzo[b][1,4]oxazinyl, 2-azaspiro[3.3]heptan-2-yl, 2-azaspiro[3.5]nonan-2-yl, 2-oxa-5-azabicyclo[2.2.1]heptanyl, 2-oxa-6-azaspiro[3.3]heptanyl, 3,4a,5,6,7,7a-hexahydro-2H-cyclopenta[b][1,4]oxazinyl, 3-oxa-8-azabicyclo[3.2.1]octanyl, 3-oxa-9-azabicyclo[3.3.1]nonanyl, 6-oxa-9-azaspiro[4.5]decanyl, 7-oxa-2-azaspiro[3.5]nonanyl, 8-oxa-3-azabicyclo[3.2.1]octanyl, azetidinyl, morpholinyl, oxan-4-yl and thiomorpholinyl. A specific group is morpholinyl.

The term “morpholino moiety is bridged by a —(CH₂)₁₋₃— moiety” means the following

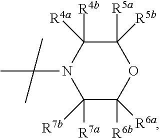

wherein all residues R^{4a-7b} are hydrogen, except

i) R^{4b} together with R^{6b} forms a —(CH₂)₁₋₃— moiety,

ii) R^{4b} together with R^{7b} forms a —(CH₂)₁₋₃— moiety,

iii) R^{5b} together with R^{6b} forms a —(CH₂)₁₋₃— moiety,

iv) R^{5b} together with R^{7b} forms a —(CH₂)₁₋₃— moiety.

Terms such as “R^{x} together with R^{y} is —(Z)ᵤ—” mean “R^{x}—(Z)ᵤ—R^{y}”, whereby x<y.

The term “pharmaceutically acceptable salts” refers to salts that are suitable for use in contact with the tissues of humans and animals. Examples of suitable salts with inorganic and organic acids are, but are not limited to acetic acid, citric acid, formic acid, fumaric acid, hydrochloric acid, lactic acid, maleic acid, malic acid, methane-sulfonic acid, nitric acid, phosphoric acid, p-toluenesulphonic acid, succinic acid, sulfuric acid (sulphuric acid), tartaric acid, trifluoroacetic acid and the like. Particular acids are formic acid, trifluoroacetic acid and hydrochloric acid.

The terms “pharmaceutically acceptable carrier” and “pharmaceutically acceptable auxiliary substance” refer to carriers and auxiliary substances such as diluents or excipients that are compatible with the other ingredients of the formulation.

The term “pharmaceutical composition” encompasses a product comprising specified ingredients in pre-determined amounts or proportions, as well as any product that results, directly or indirectly, from combining specified ingredients in specified amounts. Particularly it encompasses a product comprising one or more active ingredients, and an optional carrier comprising inert ingredients, as well as any product that results, directly or indirectly, from combination, complexation or aggregation of any two or more of the ingredients, or from dissociation of one or more of the ingredients, or from other types of reactions or interactions of one or more of the ingredients.

The term “inhibitor” denotes a compound which competes with, reduces or prevents the binding of a particular ligand to particular receptor or which reduces or prevents the inhibition of the function of a particular protein.

The term “half maximal inhibitory concentration” (IC₅₀) denotes the concentration of a particular compound required for obtaining 50% inhibition of a biological process in vitro. IC₅₀ values can be converted logarithmically to pIC₅₀ values (−log IC₅₀), in which higher values indicate exponentially greater potency. The IC₅₀ value is not an absolute value but depends on experimental conditions e.g. concentrations employed. The IC₅₀ value can be converted to an absolute inhibition constant (Ki) using the Cheng-Prusoff equation (Biochem. Pharmacol. (1973) 22:3099). The term “inhibition constant” (Ki) denotes the absolute binding affinity of a particular inhibitor to a receptor. It is measured using competition binding assays and is equal to the concentration where the particular inhibitor would occupy 50% of the receptors if no competing ligand (e.g. a radioligand) was present. Ki values can be converted logarithmically to pKi values (−log Ki), in which higher values indicate exponentially greater potency.

“Therapeutically effective amount” means an amount of a compound that, when administered to a subject for treating a disease state, is sufficient to effect such treatment for the disease state. The “therapeutically effective amount” will vary depending on the compound, disease state being treated, the severity or the disease treated, the age and relative health of the subject, the route and form of administration, the judgment of the attending medical or veterinary practitioner, and other factors.

The term “as defined herein” and “as described herein” when referring to a variable incorporates by reference the broad definition of the variable as well as particularly, more particularly and most particularly definitions, if any.

The terms “treating”, “contacting” and “reacting” when referring to a chemical reaction means adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The term “aromatic” denotes the conventional idea of aromaticity as defined in the literature, in particular in IUPAC—Compendium of Chemical Terminology, 2nd, A. D. McNaught & A. Wilkinson (Eds). Blackwell Scientific Publications, Oxford (1997).

The term “pharmaceutically acceptable excipient” denotes any ingredient having no therapeutic activity and being non-toxic such as disintegrators, binders, fillers, solvents, buffers, tonicity agents, stabilizers, antioxidants, surfactants or lubricants used in formulating pharmaceutical products.

Whenever a chiral carbon is present in a chemical structure, it is intended that all stereoisomers associated with that chiral carbon are encompassed by the structure as pure stereoisomers as well as mixtures thereof.

The invention also provides pharmaceutical compositions, methods of using, and methods of preparing the aforementioned compounds.

All separate embodiments may be combined.

E1: One embodiment of the invention provides a compound of formula I,

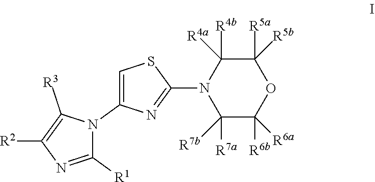

wherein
R¹ is selected from the group consisting of
i) hydrogen,
ii) halogen,
iii) CN,
iv) —C(═O)O—C₁₋₆-alkyl,
v) hydroxy-C₁₋₆-alkyl, and
vi) —X₀₋₁—C₁₋₆-alkyl;
   R² is selected from the group consisting of
i) aryl,
ii) hydrogen,
iii) halogen,
iv) CN,
v) hydroxy-C₁₋₆-alkyl, and
vi) C₁₋₆-alkyl;
   R³ is selected from the group consisting of
i) hydrogen,
ii) halogen,
iii) hydroxy-C₁₋₆-alkyl, and
iv) C₁₋₆-alkyl;
   R^{4a} is selected from the group consisting of
i) hydrogen,
ii) C₃₋₇-cycloalkyl, and
iii) C₁₋₆-alkyl;
   R^{4b} is hydrogen or —(CH₂)₁₋₄— together with
i) R^{5b},
ii) R^{6b}, or
iii) R^{7b};
   R^{5a} is selected from the group consisting of
i) aryl,
ii) heteroaryl,
iii) hydrogen,
iv) halogen-C₁₋₆-alkyl, and
v) C₁₋₆-alkyl; or
vi) together with R^{5b} is
   a. —(CH₂)₁₋₄—, or
   b. —(CH₂)₁₋₂—O—(CH₂)₁₋₂—;
      R^{5b} is selected from the group consisting of
i) hydrogen, and
ii) C₁₋₆-alkyl; or
iii) together with R^{5a} is
   a. —(CH₂)₁₋₄—, or
   b. —(CH₂)₁₋₂—O—(CH₂)₁₋₂—; or
iv) together with R^{4b} is —(CH₂)₁₋₄—, or
v) together with R^{6b} is —(CH₂)₁₋₄—, or,
vi) together with R^{7b} is —(CH₂)₁₋₄—;
   R^{6a} is selected from the group consisting of
i) hydrogen, and
ii) C₁₋₆-alkyl;
   R^{6b} is selected from the group consisting of
i) hydrogen, and
ii) C₁₋₆-alkyl; or
iii) —(CH₂)₁₋₄— together with
   a. R^{4b}, or
   b. R^{5b};
      R^{7a} is hydrogen;
      R^{7b} is selected from the group consisting of
i) hydrogen, and
ii) —(CH₂)₁₋₄— together with R^{4b}
   X is selected from the group consisting of
i) S, and
ii) O;
   or pharmaceutically acceptable salts thereof.

E2: A certain embodiment of the invention provides the compound of formula I, or a pharmaceutically acceptable salt thereof, as described herein, wherein

R¹ is selected from the group consisting of
i) hydrogen,
ii) Br,
iii) CN,
iv) —C(═O)O—CH₂CH₃,
v) hydroxy-CH₂—, and
vi) —S—CH₃,
vii) —O—CH₃, and
viii) CH₃.

E3: A certain embodiment of the invention provides the compound of formula I, or a pharmaceutically acceptable salt thereof, as described herein, wherein R¹ is Br.

E4: A certain embodiment of the invention provides the compound of formula I, or a pharmaceutically acceptable salt thereof, as described herein, wherein R² is selected from the group consisting of
i) phenyl,
ii) hydrogen,
iii) Br,
iv) Cl,
v) I,
vi) CN,
vii) hydroxy-CH₂—, and
viii) CH₃;

E5: A certain embodiment of the invention provides the compound of formula I, or a pharmaceutically acceptable salt thereof, as described herein, wherein R¹ is Br and R² is Br.

E6: A certain embodiment of the invention provides the compound of formula I, or a pharmaceutically acceptable salt thereof, as described herein, wherein R¹ is Br and R³ is Br.

E7: A certain embodiment of the invention provides the compound of formula I, or a pharmaceutically acceptable salt thereof, as described herein, wherein R² is selected from the group consisting of
i) phenyl,
ii) hydrogen,
iii) CN,
iv) hydroxy-CH₂—, and
v) CH₃.

E8: A certain embodiment of the invention provides the compound of formula I, or a pharmaceutically acceptable salt thereof, as described herein, wherein R² is Br.

E9: A certain embodiment of the invention provides the compound of formula I, or a pharmaceutically acceptable salt thereof, as described herein, wherein

R³ is selected from the group consisting of
i) hydrogen,
ii) Br,
iii) Cl,
iv) I,
v) hydroxy-CH₂—, and
vi) CH₃;
   E10: A certain embodiment of the invention provides the compound of formula I, or a pharmaceutically acceptable salt thereof, as described herein, wherein R³ is hydrogen.

E11: A certain embodiment of the invention provides the compound of formula I, or a pharmaceutically acceptable salt thereof, as described herein, wherein the morpholino moiety is bridged by a —(CH₂)₁₋₃— moiety.

E12: A certain embodiment of the invention provides the compound of formula I, or a pharmaceutically acceptable salt thereof, as described herein, that is selected from the group consisting of
(1-(2-((1R,5S)-3-oxa-8-azabicyclo[3.2.1]octan-8-yl)thiazol-4-yl)-4-chloro-1H-imidazol-2-yl)methanol,
(1R,5S)-3-[4-(4,5-dibromoimidazol-1-yl)thiazol-2-yl]-8-oxa-3-azabicyclo[3.2.1]octane,
(1R,5S)-3-[4-(4-bromo-5-chloro-imidazol-1-yl)thiazol-2-yl]-8-oxa-3-azabicyclo[3.2.1]octane,
(1R,5S)-3-[4-(5-bromo-4-chloro-imidazol-1-yl)thiazol-2-yl]-8-oxa-3-azabicyclo[3.2.1]octane,
(1R,5S)-8-(4-(4-chloro-1H-imidazol-1-yl)thiazol-2-yl)-3-oxa-8-azabicyclo[3.2.1]octane,
(1R,5S)-8-[4-(2,4-dibromoimidazol-1-yl)thiazol-2-yl]-3-oxa-8-azabicyclo[3.2.1]octane,
(1R,5S)-8-[4-(4,5-dibromoimidazol-1-yl)thiazol-2-yl]-3-oxa-8-azabicyclo[3.2.1]octane,
(1R,5S)-8-[4-(4,5-dichloroimidazol-1-yl)thiazol-2-yl]-3-oxa-8-azabicyclo[3.2.1]octane,
(1R,5S)-8-[4-(4-bromo-2-methylsulfanyl-imidazol-1-yl)thiazol-2-yl]-3-oxa-8-azabicyclo[3.2.1]octane,
(1R,5S)-8-[4-(4-bromo-5-chloro-imidazol-1-yl)thiazol-2-yl]-3-oxa-8-azabicyclo[3.2.1]octane,
(1R,5S)-8-[4-(5-bromo-4-chloro-imidazol-1-yl)thiazol-2-yl]-3-oxa-8-azabicyclo[3.2.1]octane,
(1R,5S)-9-[4-(4-bromo-5-chloro-imidazol-1-yl)thiazol-2-yl]-3-oxa-9-azabicyclo[3.3.1]nonane,
(1R,5S)-9-[4-(5-bromo-4-chloro-imidazol-1-yl)thiazol-2-yl]-3-oxa-9-azabicyclo[3.3.1]nonane,
(1S,4S)-5-[4-(2,4-dibromoimidazol-1-yl)thiazol-2-yl]-2-oxa-5-azabicyclo[2.2.1]heptane,
(1S,4S)-5-[4-(4,5-dibromoimidazol-1-yl)thiazol-2-yl]-2-oxa-5-azabicyclo[2.2.1]heptane,
(1S,5R)-3-[4-(2,4-dibromoimidazol-1-yl)thiazol-2-yl]-8-oxa-3-azabicyclo[3.2.1]octane,
(1S,5R)-3-[4-(4,5-dichloroimidazol-1-yl)thiazol-2-yl]-8-oxa-3-azabicyclo[3.2.1]octane, (1S,5R)-8-[4-(4-bromo-2-methoxy-imidazol-1-yl)thiazol-2-yl]-3-oxa-8-azabicyclo[3.2.1]octane,
(1S,5R)-9-[4-(2,4-dibromoimidazol-1-yl)thiazol-2-yl]-3-oxa-9-azabicyclo[3.3.1]nonane,
(1S,5R)-9-[4-(4,5-dibromoimidazol-1-yl)thiazol-2-yl]-3-oxa-9-azabicyclo[3.3.1]nonane,
(2-((1R,5S)-3-oxa-8-azabicyclo[3.2.1]octan-8-yl)-4-(4-chloro-1H-imidazol-1-yl)thiazol-5-yl)methanol,
[4,5-dibromo-1-[2-[(1R,5S)-3-oxa-8-azabicyclo[3.2.1]octan-8-yl]thiazol-4-yl]imidazol-2-yl]methanol,
[5-bromo-1-(2-morpholinothiazol-4-yl)imidazol-4-yl]methanol,
[5-bromo-3-(2-morpholinothiazol-4-yl)imidazol-4-yl]methanol,
1-(2-morpholinothiazol-4-yl)imidazole-4-carbonitrile,
2,6-ditert-butyl-4-[4-(2,4-dibromoimidazol-1-yl)thiazol-2-yl]morpholine,
2-benzyl-4-[4-(2,4-dibromoimidazol-1-yl)thiazol-2-yl]morpholine,
3-cyclopropyl-4-[4-(2,4-dibromoimidazol-1-yl)thiazol-2-yl]morpholine,
4-(4-(4,5-dibromo-1H-imidazol-1-yl)thiazol-2-yl)-2,2-diethylmorpholine,
4-(4-imidazol-1-ylthiazol-2-yl)morpholine,
4-[4-(2,4-dibromoimidazol-1-yl)thiazol-2-yl]-1,9-dioxa-4-azaspiro[5.5]undecane,
4-[4-(2,4-dibromoimidazol-1-yl)thiazol-2-yl]-2-(2-thienyl)morpholine,
4-[4-(2,4-dibromoimidazol-1-yl)thiazol-2-yl]-2-(trifluoromethyl)morpholine,
4-[4-(2,4-dibromoimidazol-1-yl)thiazol-2-yl]-2-(trifluoromethyl)morpholine,
4-[4-(2,4-dibromoimidazol-1-yl)thiazol-2-yl]-2,2-diethyl-morpholine,
4-[4-(2,4-dibromoimidazol-1-yl)thiazol-2-yl]-2,2-dimethyl-morpholine,
4-[4-(2,4-dibromoimidazol-1-yl)thiazol-2-yl]-2,3,4a,5,6,7,8,8a-octahydrobenzo[b][1,4]oxazine,
4-[4-(2,4-dibromoimidazol-1-yl)thiazol-2-yl]-2,5-dimethyl-morpholine,
4-[4-(2,4-dibromoimidazol-1-yl)thiazol-2-yl]-2-isopropyl-morpholine,
4-[4-(2,4-dibromoimidazol-1-yl)thiazol-2-yl]-2-methyl-morpholine,
4-[4-(2,4-dibromoimidazol-1-yl)thiazol-2-yl]-2-phenyl-morpholine,
4-[4-(2,4-dibromoimidazol-1-yl)thiazol-2-yl]-3,4a,5,6,7,7a-hexahydro-2H-cyclopenta[b][1,4]oxazine,
4-[4-(2,4-dibromoimidazol-1-yl)thiazol-2-yl]-3-ethyl-morpholine,
4-[4-(2,4-dibromoimidazol-1-yl)thiazol-2-yl]morpholine,
4-[4-(2-bromoimidazol-1-yl)thiazol-2-yl]morpholine,
4-[4-(2-methyl-4-phenyl-imidazol-1-yl)thiazol-2-yl]morpholine,
4-[4-(4,5-dibromoimidazol-1-yl)thiazol-2-yl]-2,6-dimethyl-morpholine
4-[4-(2,4-dibromoimidazol-1-yl)thiazol-2-yl]-2,6-dimethyl-morpholine,
4-[4-(4,5-dibromoimidazol-1-yl)thiazol-2-yl]morpholine,
4-[4-(4,5-dichloroimidazol-1-yl)thiazol-2-yl]morpholine,
4-[4-(4,5-diiodoimidazol-1-yl)thiazol-2-yl]morpholine,
4-[4-(4,5-dimethylimidazol-1-yl)thiazol-2-yl]morpholine,
4-[4-(4-bromo-2-methyl-imidazol-1-yl)thiazol-2-yl]morpholine,
4-[4-(4-bromo-2-methylsulfanyl-imidazol-1-yl)thiazol-2-yl]morpholine,
4-[4-(4-bromo-5-chloro-imidazol-1-yl)thiazol-2-yl]morpholine,
4-[4-(4-bromo-5-methyl-imidazol-1-yl)thiazol-2-yl]morpholine,
4-[4-(4-bromoimidazol-1-yl)thiazol-2-yl]morpholine,
4-[4-(4-chloro-5-iodo-imidazol-1-yl)thiazol-2-yl]morpholine,
4-[4-(4-chloroimidazol-1-yl)thiazol-2-yl]morpholine,
4-[4-(5-bromo-4-chloro-imidazol-1-yl)thiazol-2-yl]morpholine,
4-[4-(5-bromo-4-methyl-imidazol-1-yl)thiazol-2-yl]morpholine,
4-bromo-1-(2-morpholinothiazol-4-yl)imidazole-2-carbonitrile,
5-[4-(2,4-dibromoimidazol-1-yl)thiazol-2-yl]-2-oxa-5-azabicyclo[2.2.1]heptane,
8-(4-(4,5-dibromo-1H-imidazol-1-yl)thiazol-2-yl)-2,5-dioxa-8-azaspiro[3.5]nonane,
8-[4-(2,4-dibromoimidazol-1-yl)thiazol-2-yl]-2,5-dioxa-8-azaspiro[3.5]nonane,
9-[4-(2,4-dibromoimidazol-1-yl)thiazol-2-yl]-6-oxa-9-azaspiro[4.5]decane,
ethyl 4,5-dibromo-1-(2-morpholinothiazol-4-yl)imidazole-2-carboxylate, and
ethyl 4,5-dibromo-1-(2-morpholinothiazol-4-yl)imidazole-2-carboxylate,
   or pharmaceutically acceptable salts thereof.

E13: A certain embodiment of the invention provides the compound of formula I, or a pharmaceutically acceptable salt thereof, as described herein, that is selected from the group consisting of
(1-(2-((1R,5S)-3-oxa-8-azabicyclo[3.2.1]octan-8-yl)thiazol-4-yl)-4-chloro-1H-imidazol-2-yl)methanol,
(1R,5S)-3-[4-(4,5-dibromoimidazol-1-yl)thiazol-2-yl]-8-oxa-3-azabicyclo[3.2.1]octane,
(1R,5S)-3-[4-(4-bromo-5-chloro-imidazol-1-yl)thiazol-2-yl]-8-oxa-3-azabicyclo[3.2.1]octane,
(1R,5S)-3-[4-(5-bromo-4-chloro-imidazol-1-yl)thiazol-2-yl]-8-oxa-3-azabicyclo[3.2.1]octane,
(1R,5S)-8-(4-(4-chloro-1H-imidazol-1-yl)thiazol-2-yl)-3-oxa-8-azabicyclo[3.2.1]octane,
(1R,5S)-8-[4-(2,4-dibromoimidazol-1-yl)thiazol-2-yl]-3-oxa-8-azabicyclo[3.2.1]octane,
(1R,5S)-8-[4-(4,5-dibromoimidazol-1-yl)thiazol-2-yl]-3-oxa-8-azabicyclo[3.2.1]octane,
(1R,5S)-8-[4-(4,5-dichloroimidazol-1-yl)thiazol-2-yl]-3-oxa-8-azabicyclo[3.2.1]octane,
(1R,5S)-8-[4-(4-bromo-2-methylsulfanyl-imidazol-1-yl)thiazol-2-yl]-3-oxa-8-azabicyclo[3.2.1]octane,
(1R,5S)-8-[4-(4-bromo-5-chloro-imidazol-1-yl)thiazol-2-yl]-3-oxa-8-azabicyclo[3.2.1]octane,
(1R,5S)-8-[4-(5-bromo-4-chloro-imidazol-1-yl)thiazol-2-yl]-3-oxa-8-azabicyclo[3.2.1]octane,
(1R,5S)-9-[4-(4-bromo-5-chloro-imidazol-1-yl)thiazol-2-yl]-3-oxa-9-azabicyclo[3.3.1]nonane,
(1R,5S)-9-[4-(5-bromo-4-chloro-imidazol-1-yl)thiazol-2-yl]-3-oxa-9-azabicyclo[3.3.1]nonane,
(1S,4S)-5-[4-(2,4-dibromoimidazol-1-yl)thiazol-2-yl]-2-oxa-5-azabicyclo[2.2.1]heptane,
(1S,4S)-5-[4-(4,5-dibromoimidazol-1-yl)thiazol-2-yl]-2-oxa-5-azabicyclo[2.2.1]heptane,
(1S,5R)-3-[4-(2,4-dibromoimidazol-1-yl)thiazol-2-yl]-8-oxa-3-azabicyclo[3.2.1]octane,
(1S,5R)-3-[4-(4,5-dichloroimidazol-1-yl)thiazol-2-yl]-8-oxa-3-azabicyclo[3.2.1]octane,
(1S,5R)-8-[4-(4-bromo-2-methoxy-imidazol-1-yl)thiazol-2-yl]-3-oxa-8-azabicyclo[3.2.1]octane,
(1S,5R)-9-[4-(2,4-dibromoimidazol-1-yl)thiazol-2-yl]-3-oxa-9-azabicyclo[3.3.1]nonane,
(1S,5R)-9-[4-(4,5-dibromoimidazol-1-yl)thiazol-2-yl]-3-oxa-9-azabicyclo[3.3.1]nonane,
(2-((1R,5S)-3-oxa-8-azabicyclo[3.2.1]octan-8-yl)-4-(4-chloro-1H-imidazol-1-yl)thiazol-5-yl)methanol,
[4,5-dibromo-1-[2-[(1R,5S)-3-oxa-8-azabicyclo[3.2.1]octan-8-yl]thiazol-4-yl]imidazol-2-yl]methanol, and
5-[4-(2,4-dibromoimidazol-1-yl)thiazol-2-yl]-2-oxa-5-azabicyclo[2.2.1]heptane,
   or pharmaceutically acceptable salts thereof.

E13: A certain embodiment of the invention provides the compound of formula I, or a pharmaceutically acceptable salt thereof, as described herein, that is 4-[4-(2,4-dibromoimidazol-1-yl)thiazol-2-yl]morpholino.

E14: A certain embodiment of the invention provides the compound of formula I, or a pharmaceutically acceptable salt thereof, as described herein, whenever prepared by a process as defined herein.

E15: A certain embodiment of the invention provides the compound of formula I, or a pharmaceutically acceptable salt thereof, as described herein for use as therapeutically active substance.

E16: A certain embodiment of the invention provides the compound of formula I, or a pharmaceutically acceptable salt thereof, as described herein for the use as inhibitor of AR-V7.

E17: A certain embodiment of the invention provides the compound of formula I, or a pharmaceutically acceptable salt thereof, as described herein for the use as therapeutically active substance for the therapeutic and/or prophylactic treatment of cancers, in particular prostate cancer, breast cancer, bladder cancer, endometrial and ovarian cancers, hepatocellular and pancreatic cancers, and salivary gland cancers.

E18: A certain embodiment of the invention provides the compound of formula I, or a pharmaceutically acceptable salt thereof, as described herein for the use as therapeutically active substance for the treatment of cancers, in particular prostate cancer, breast cancer, bladder cancer, endometrial and ovarian cancers, hepatocellular and pancreatic cancers, and salivary gland cancers.

E19: A certain embodiment of the invention provides the compound of formula I, or a pharmaceutically acceptable salt thereof, as described herein for the use as therapeutically active substance for the treatment of prostate cancer, in particular metastatic castration-resistant prostate cancer.

E20: A certain embodiment of the invention provides the compound of formula I, or a pharmaceutically acceptable salt thereof, as described herein and a pharmaceutically acceptable carrier and/or a pharmaceutically acceptable auxiliary substance.

E21: A certain embodiment of the invention provides the compound of formula I, or a pharmaceutically acceptable salt thereof, as described herein for the manufacture of a medicament for the use in inhibition of AR-V7.

E22: A certain embodiment of the invention provides the use of a compound of formula I, or a pharmaceutically acceptable salt thereof, as described herein for the manufacture of a medicament for the therapeutically active substance for the therapeutic and/or prophylactic treatment of cancers, in particular prostate cancer, breast cancer, bladder cancer, endometrial and ovarian cancers, hepatocellular and pancreatic cancers, and salivary gland cancers.

E23: A certain embodiment of the invention provides a method for the use of a compound of formula I, or a pharmaceutically acceptable salt thereof, as described herein in inhibition of AR-V7 activity, particularly for the therapeutically active substance for the therapeutic and/or prophylactic treatment of cancers, in particular prostate cancer, breast cancer, bladder cancer, endometrial and ovarian cancers, hepatocellular and pancreatic cancers, and salivary gland cancers.

E24: A certain embodiment of the invention provides of a compound of formula I, or a pharmaceutically acceptable salt thereof, as described herein for the use as a medicament in the treatment of an AR-V7-positive patient suffering from cancer, in particular prostate cancer, comprising determining the AR-V7-status in said patient and administering a compound of formula I as described herein to said patient.

Furthermore, the invention includes all optical isomers, i.e. diastereoisomers, diastereomeric mixtures, racemic mixtures, all their corresponding enantiomers and/or tautomers as well as their solvates of the compounds of formula I.

The compounds of formula I may contain one or more asymmetric centers and can therefore occur as racemates, racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within this invention. The present invention is meant to encompass all such isomeric forms of these compounds. The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography.

In the embodiments, where optically pure enantiomers are provided, optically pure enantiomer means that the compound contains >90% of the desired isomer by weight, particularly >95% of the desired isomer by weight, or more particularly >99% of the desired isomer by weight, said weight percent based upon the total weight of the isomer(s) of the compound. Chirally pure or chirally enriched compounds may be prepared by chirally selective synthesis or by separation of enantiomers. The separation of enantiomers may be carried out on the final product or alternatively on a suitable intermediate.

The compounds of formula I may be prepared in accordance with the following schemes. The starting material is commercially available or may be prepared in accordance with known methods. Any previously defined residues and variables will continue to have the previously defined meaning unless otherwise indicated.

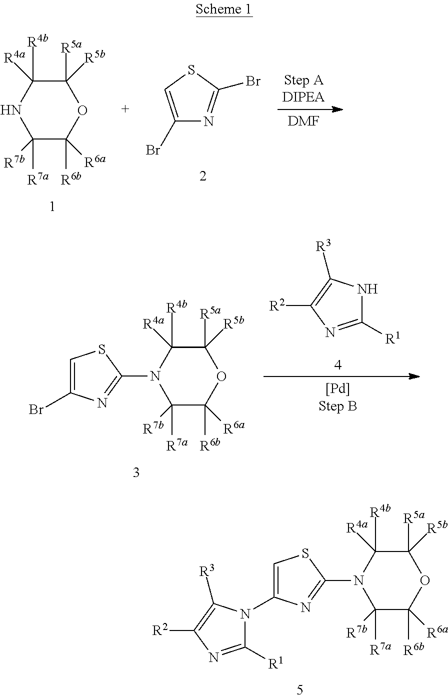

wherein R¹-R^{7b} have the meanings as herein described
   Steps A and B can take place in a one-pot procedure.

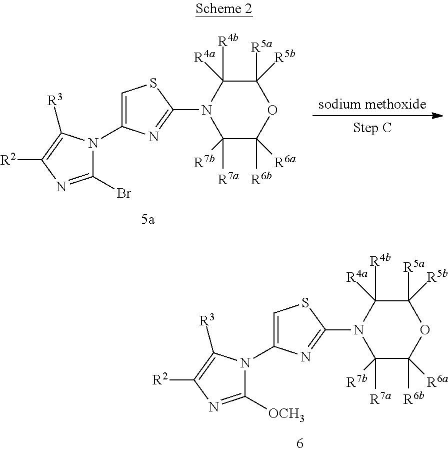

wherein R¹-R^{7b} have the meanings as herein described

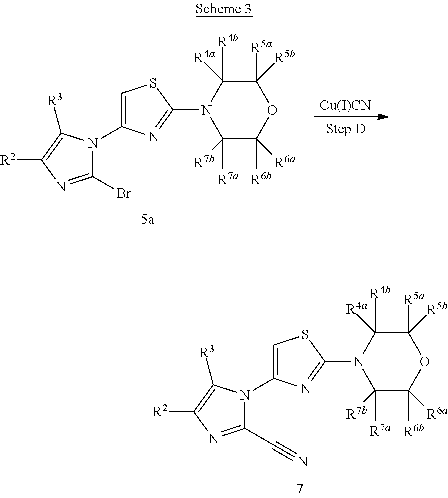

wherein R¹-R^{7b} have the meanings as herein described

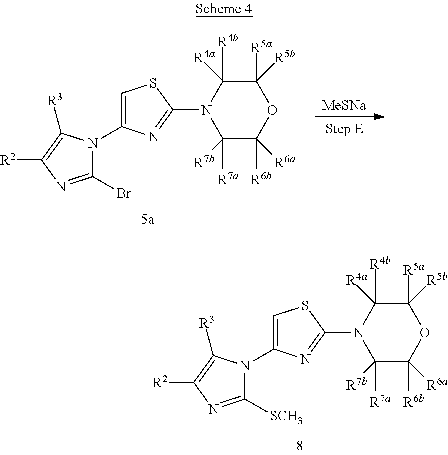

wherein R¹-R^{7b} have the meanings as herein described

The corresponding pharmaceutically acceptable salts with acids can be obtained by standard methods known to the person skilled in the art, e.g. by dissolving the compound of formula I in a suitable solvent such as e.g. dioxane or tetrahydrofuran and adding an appropriate amount of the corresponding acid. The products can usually be isolated by filtration or by chromatography. The conversion of a compound of formula I into a pharmaceutically acceptable salt with a base can be carried out by treatment of such a compound with such a base. One possible method to form such a salt is e.g. by addition of 1/n equivalents of a basic salt such as e.g. M(OH)ₙ, wherein M=metal or ammonium cation and n=number of hydroxide anions, to a solution of the compound in a suitable solvent (e.g. ethanol, ethanol-water mixture, tetrahydrofuran-water mixture) and to remove the solvent by evaporation or lyophilisation. Particular salts are hydrochloride, formate and trifluoroacetate. A specific salt is trifluoroacetate.

Insofar as their preparation is not described in the examples, the compounds of formula I as well as all intermediate products can be prepared according to analogous methods or according to the methods set forth herein. Starting materials are commercially available, known in the art or can be prepared by methods known in the art or in analogy thereto.

It will be appreciated that the compounds of general formula I in this invention may be derivatised at functional groups to provide derivatives which are capable of conversion back to the parent compound in vivo.

### Pharmacological Tests

The compounds of formula I and their pharmaceutically acceptable salts possess valuable pharmacological properties. It has been found that the compounds of the present invention are associated with inhibition of AR-V7 activity. The compounds were investigated in accordance with the test given hereinafter.

The DNA-binding domain is an attractive target for inhibition of AR dimerization and/or DNA binding. In silico computational drug discovery methods were used to conduct a virtual screen of >3 million purchasable lead-like compounds from the ZINC database (Irwin, J. et al.¹¹) to identify potential DBD binders. The in silico methods included large-scale docking, in-site rescoring and consensus voting procedures. It will be understood by a person of skill that COOH and NR₂ may include the corresponding ions, for example carboxylate ions and ammonium ions, respectively. Alternatively, where the ions are shown, a person of skill in the art will appreciate that the counter ion may also be present. Furthermore, it will be appreciated by a person of skill that other moieties may include the corresponding ions, and where the ions are shown, a person of skill in the art will appreciate that the counter ion may also be present. The table below shows the compounds tested by structure and the associated identifiers. Where the % inhibition, eGFP IC₅₀ or the PSA IC₅₀ has no value given, this may be because no measurement was taken or the value was not calculated, in the case of the % inhibition. In vitro identification of AR inhibitors targeting AR DBD The selected compounds were assessed with a non-destructive eGFP assay that quantifies levels of AR transcriptional activity, were identified (table below).

TABLE 1
IC₅₀ value

| | | | eGFP | PSA |
|---|---|---|---|---|
| | | | IC₅₀ | IC₅₀ |
| Ex. | Structure | Name | [nM] | [μM] |
| 1 | 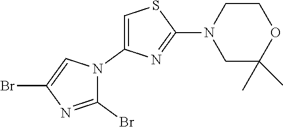 [SMILES: CC1(C)CN(c2nc(-n3cc(Br)nc3Br)cs2)CCO1] | 4-[4-(2,4-dibromoimidazol-1- yl)thiazol-2-yl]-2,2-dimethyl- morpholine | 117 | 95 |
| 2 | 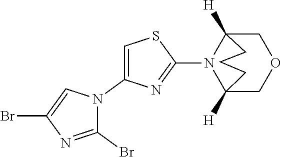 [SMILES: Brc1cn(-c2csc(N3[C@@H]4CCC[C@H]3COC4)n2)c(Br)n1] | (1S,5R)-9-[4-(2,4- dibromoimidazol-1-yl)thiazol- 2-yl]-3-oxa-9- azabicyclo[3.3.1]nonane | 135 | 74 |
| 3 | 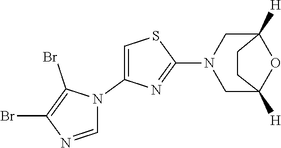 [SMILES: Brc1ncn(-c2csc(N3C[C@H]4CC[C@@H](C3)O4)n2)c1Br] | (1R,5S)-3-[4-(4,5- dibromoimidazol-1-yl)thiazol- 2-yl]-8-oxa-3- azabicyclo[3.2.1]octane | 182 | 1062 |
| 4 | 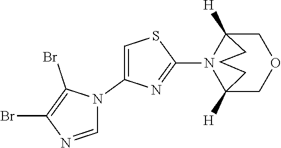 [SMILES: Brc1ncn(-c2csc(N3[C@@H]4CCC[C@H]3COC4)n2)c1Br] | (1S,5R)-9-[4-(4,5- dibromoimidazol-1-yl)thiazol- 2-yl]-3-oxa-9- azabicyclo[3.3.1]nonane | 196 | 6 |
| 5 | 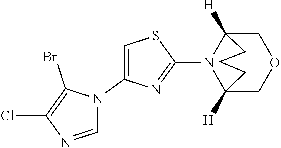 [SMILES: Clc1ncn(-c2csc(N3[C@@H]4CCC[C@H]3COC4)n2)c1Br] | (1R,5S)-9-[4-(5-bromo-4- chloro-imidazol-1-yl)thiazol-2- yl]-3-oxa-9- azabicyclo[3.3.1]nonane | 268 | 181 |
| 6 | 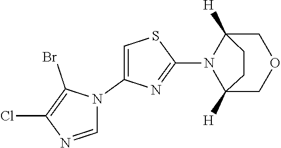 [SMILES: Clc1ncn(-c2csc(N3[C@@H]4CC[C@H]3COC4)n2)c1Br] | (1R,5S)-8-[4-(5-bromo-4- chloro-imidazol-1-yl)thiazol-2- yl]-3-oxa-8- azabicyclo[3.2.1]octane | 404 | 377 |
| 7 | 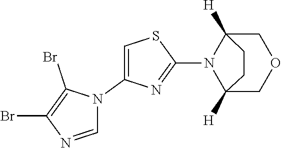 [SMILES: Brc1ncn(-c2csc(N3[C@@H]4CC[C@H]3COC4)n2)c1Br] | (1R,5S)-8-[4-(4,5- dibromoimidazol-1-yl)thiazol- 2-yl]-3-oxa-8- azabicyclo[3.2.1]octane | 458 | 385 |
| 8 | 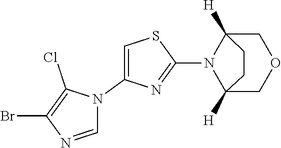 [SMILES: Clc1c(Br)ncn1-c1csc(N2[C@@H]3CC[C@H]2COC3)n1] | (1R,5S)-8-[4-(4-bromo-5- chloro-imidazol-1-yl)thiazol-2- yl]-3-oxa-8- azabicyclo[3.2.1]octane | 717 | 717 |
| 9 | 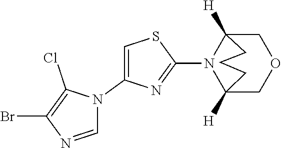 [SMILES: Clc1c(Br)ncn1-c1csc(N2[C@@H]3CCC[C@H]2COC3)n1] | (1R,5S)-9-[4-(4-bromo-5- chloro-imidazol-1-yl)thiazol-2- yl]-3-oxa-9- azabicyclo[3.3.1]nonane | 746 | 784 |
| 10 | 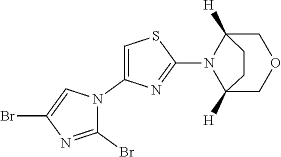 [SMILES: Brc1cn(-c2csc(N3[C@@H]4CC[C@H]3COC4)n2)c(Br)n1] | (1R,5S)-8-[4-(2,4- dibromoimidazol-1-yl)thiazol- 2-yl]-3-oxa-8- azabicyclo[3.2.1]octane | 934 | 817 |
| 11 | 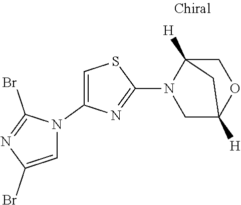 [SMILES: Brc1cn(-c2csc(N3C[C@@H]4C[C@H]3CO4)n2)c(Br)n1] | (1S,4S)-5-[4-(2,4- dibromoimidazol-1-yl)thiazol- 2-yl]-2-oxa-5- azabicyclo[2.2.1]heptane | 1181 | |
| 12 | 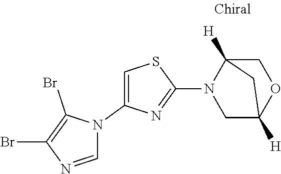 [SMILES: Brc1ncn(-c2csc(N3C[C@@H]4C[C@H]3CO4)n2)c1Br] | (1S,4S)-5-[4-(4,5- dibromoimidazol-1-yl)thiazol- 2-yl]-2-oxa-5- azabicyclo[2.2.1]heptane | 1322 | |
| 13 | 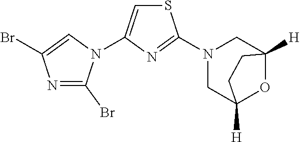 [SMILES: Brc1cn(-c2csc(N3C[C@H]4CC[C@@H](C3)O4)n2)c(Br)n1] | (1S,5R)-3-[4-(2,4- dibromoimidazol-1-yl)thiazol- 2-yl]-8-oxa-3- azabicyclo[3.2.1]octane | 110 | |
| 14 | 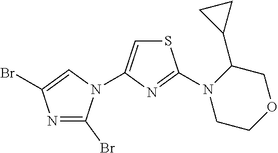 [SMILES: Brc1cn(-c2csc(N3CCOCC3C3CC3)n2)c(Br)n1] | 3-cyclopropyl-4-[4-(2,4- dibromoimidazol-1-yl)thiazol- 2-yl]morpholine | 120 | |
| 15 | 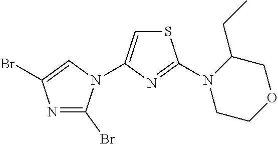 [SMILES: CCC1COCCN1c1nc(-n2cc(Br)nc2Br)cs1] | 4-[4-(2,4-dibromoimidazol-1- yl)thiazol-2-yl]-3-ethyl- morpholine | 130 | |
| 16 | 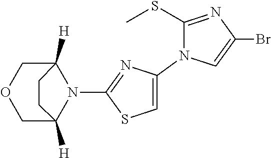 [SMILES: CSc1nc(Br)cn1-c1csc(N2[C@@H]3CC[C@H]2COC3)n1] | (1R,5S)-8-[4-(4-bromo-2- methylsulfanyl-imidazol-1- yl)thiazol-2-yl]-3-oxa-8- azabicyclo[3.2.1]octane | 140 | |
| 17 | 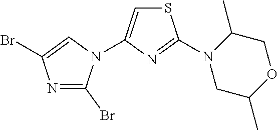 [SMILES: CC1CN(c2nc(-n3cc(Br)nc3Br)cs2)C(C)CO1] | 4-[4-(2,4-dibromoimidazol-1- yl)thiazol-2-yl]-2,5-dimethyl- morpholine | 190 | |
| 18 | 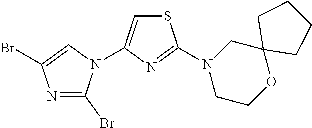 [SMILES: Brc1cn(-c2csc(N3CCOC4(CCCC4)C3)n2)c(Br)n1] | 9-[4-(2,4-dibromoimidazol-1- yl)thiazol-2-yl]-6-oxa-9- azaspiro[4.5]decane | 200 | |
| 19 | 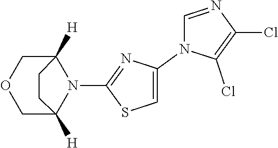 [SMILES: Clc1ncn(-c2csc(N3[C@@H]4CC[C@H]3COC4)n2)c1Cl] | (1R,5S)-8-[4-(4,5- dichloroimidazol-1-yl)thiazol- 2-yl]-3-oxa-8- azabicyclo[3.2.1]octane | 320 | |
| 20 | 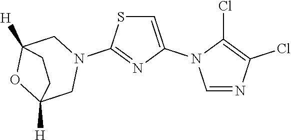 [SMILES: Clc1ncn(-c2csc(N3C[C@H]4CC[C@@H](C3)O4)n2)c1Cl] | (1S,5R)-3-[4-(4,5- dichloroimidazol-1-yl)thiazol- 2-yl]-8-oxa-3- azabicyclo[3.2.1]octane | 250 | |
| 21 | 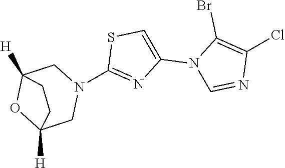 [SMILES: Clc1ncn(-c2csc(N3C[C@H]4CC[C@@H](C3)O4)n2)c1Br] | (1R,5S)-3-[4-(5-bromo-4- chloro-imidazol-1-yl)thiazol-2- yl]-8-oxa-3- azabicyclo[3.2.1]octane | 300 | |
| 22 | 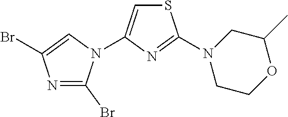 [SMILES: CC1CN(c2nc(-n3cc(Br)nc3Br)cs2)CCO1] | 4-[4-(2,4-dibromoimidazol-1- yl)thiazol-2-yl]-2-methyl- morpholine | 400 | |
| 23 | 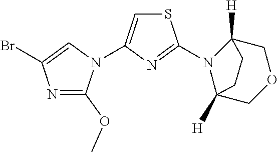 [SMILES: COc1nc(Br)cn1-c1csc(N2[C@@H]3CC[C@H]2COC3)n1] | (1S,5R)-8-[4-(4-bromo-2- methoxy-imidazol-1- yl)thiazol-2-yl]-3-oxa-8- azabicyclo[3.2.1]octane | 270 | |
| 24 | 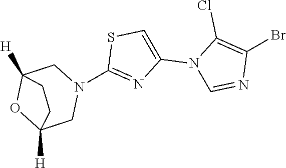 [SMILES: Clc1c(Br)ncn1-c1csc(N2C[C@H]3CC[C@@H](C2)O3)n1] | (1R,5S)-3-[4-(4-bromo-5- chloro-imidazol-1-yl)thiazol-2- yl]-8-oxa-3- azabicyclo[3.2.1]octane | 700 | |
| 25 | 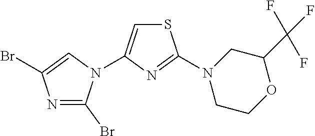 [SMILES: FC(F)(F)C1CN(c2nc(-n3cc(Br)nc3Br)cs2)CCO1] | 4-[4-(2,4-dibromoimidazol-1- yl)thiazol-2-yl]-2- (trifluoromethyl)morpholine | 1700 | |
| 26 | 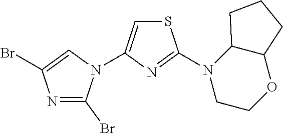 [SMILES: Brc1cn(-c2csc(N3CCOC4CCCC43)n2)c(Br)n1] | 4-[4-(2,4-dibromoimidazol-1- yl)thiazol-2-yl]-3,4a,5,6,7,7a- hexahydro-2H- cyclopenta[b][1,4]oxazine | 1530 | |
| 27 | 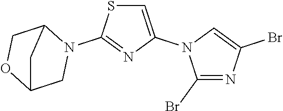 [SMILES: Brc1cn(-c2csc(N3CC4CC3CO4)n2)c(Br)n1] | 5-[4-(2,4-dibromoimidazol-1- yl)thiazol-2-yl]-2-oxa-5- azabicyclo[2.2.1]heptane | 1610 | |
| 28 | 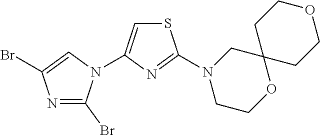 [SMILES: Brc1cn(-c2csc(N3CCOC4(CCOCC4)C3)n2)c(Br)n1] | 4-[4-(2,4-dibromoimidazol-1- yl)thiazol-2-yl]-1,9-dioxa-4- azaspiro[5.5]undecane | 3120 | |
| 29 | 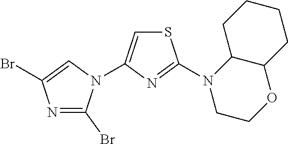 [SMILES: Brc1cn(-c2csc(N3CCOC4CCCCC43)n2)c(Br)n1] | 4-[4-(2,4-dibromoimidazol-1- yl)thiazol-2-yl]- 2,3,4a,5,6,7,8,8a- octahydro- benzo[b][1,4]oxazine | 3150 | |
| 30 | 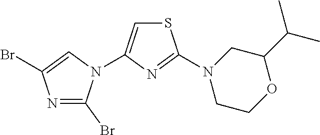 [SMILES: CC(C)C1CN(c2nc(-n3cc(Br)nc3Br)cs2)CCO1] | 4-[4-(2,4-dibromoimidazol-1- yl)thiazol-2-yl]-2-isopropyl- morpholine | 6650 | |
| 31 | 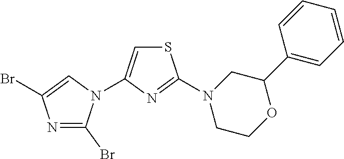 [SMILES: Brc1cn(-c2csc(N3CCOC(c4ccccc4)C3)n2)c(Br)n1] | 4-[4-(2,4-dibromoimidazol-1- yl)thiazol-2-yl]-2-phenyl- morpholine | 8150 | |
| 32 | 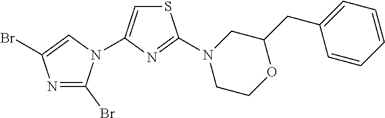 [SMILES: Brc1cn(-c2csc(N3CCOC(Cc4ccccc4)C3)n2)c(Br)n1] | 2-benzyl-4-[4-(2,4- dibromoimidazol-1-yl)thiazol- 2-yl]morpholine | 27410 | |
| 33 | 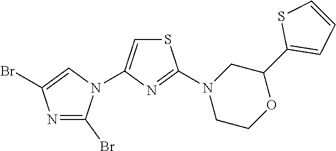 [SMILES: Brc1cn(-c2csc(N3CCOC(c4cccs4)C3)n2)c(Br)n1] | 4-[4-(2,4-dibromoimidazol-1- yl)thiazol-2-yl]-2-(2- thienyl)morpholine | 170 | |
| 34 | 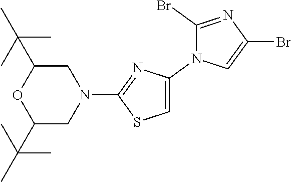 [SMILES: CC(C)(C)C1CN(c2nc(-n3cc(Br)nc3Br)cs2)CC(C(C)(C)C)O1] | 2,6-ditert-butyl-4-[4-(2,4- dibromoimidazol-1-yl)thiazol- 2-yl]morpholine | inact. | |
| 35 | 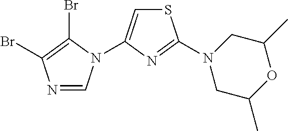 [SMILES: CC1CN(c2nc(-n3cnc(Br)c3Br)cs2)CC(C)O1] | 4-[4-(4,5-dibromoimidazol-1- yl)thiazol-2-yl]-2,6-dimethyl- morpholine | n.d. | |
| | 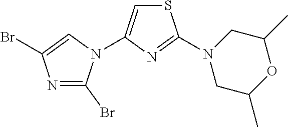 [SMILES: CC1CN(c2nc(-n3cc(Br)nc3Br)cs2)CC(C)O1] | 4-[4-(2,4-dibromoimidazol-1- yl)thiazol-2-yl]-2,6-dimethyl- morpholine | | |
| 36 | 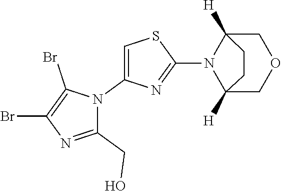 [SMILES: OCc1nc(Br)c(Br)n1-c1csc(N2[C@@H]3CC[C@H]2COC3)n1] | [4,5-dibromo-1-[2-[(1R,5S)-3- oxa-8-azabicyclo[3.2.1]octan- 8-yl]thiazol-4-yl]imidazol-2- yl]methanol | 2991 | |
| 37 | 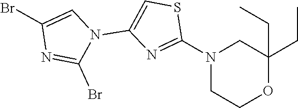 [SMILES: CCC1(CC)CN(c2nc(-n3cc(Br)nc3Br)cs2)CCO1] | 4-[4-(2,4-dibromoimidazol-1- yl)thiazol-2-yl]-2,2-diethyl- morpholine | 33% inhib. at 1 μM | |
| 38 | 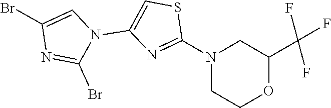 [SMILES: FC(F)(F)C1CN(c2nc(-n3cc(Br)nc3Br)cs2)CCO1] | 4-[4-(2,4-dibromoimidazol-1- yl)thiazol-2-yl]-2- (trifluoromethyl)morpholine | 1949 | |
| 39 | 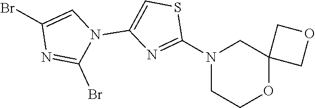 [SMILES: Brc1cn(-c2csc(N3CCOC4(COC4)C3)n2)c(Br)n1] | 8-[4-(2,4-dibromoimidazol-1- yl)thiazol-2-yl]-2,5-dioxa-8- azaspiro[3.5]nonane | 34% inhib. at 1 μM | |
| 40 | 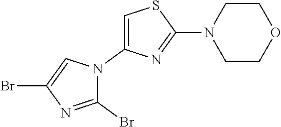 [SMILES: Brc1cn(-c2csc(N3CCOCC3)n2)c(Br)n1] | 4-[4-(2,4-dibromoimidazol-1- yl)thiazol-2-yl]morpholine | 413 | 420 |
| 41 | 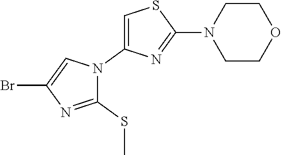 [SMILES: CSc1nc(Br)cn1-c1csc(N2CCOCC2)n1] | 4-[4-(4-bromo-2- methylsulfanyl-imidazol-1- yl)thiazol-2-yl]morpholine | 496 | 458 |
| 42 | 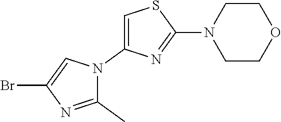 [SMILES: Cc1nc(Br)cn1-c1csc(N2CCOCC2)n1] | 4-[4-(4-bromo-2-methyl- imidazol-1-yl)thiazol-2- yl]morpholine | 750 | 659 |
| 43 | 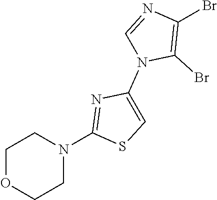 [SMILES: Brc1ncn(-c2csc(N3CCOCC3)n2)c1Br] | 4-[4-(4,5-dibromoimidazol-1- yl)thiazol-2-yl]morpholine | 1232 | 1391 |
| 44 | 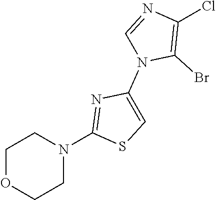 [SMILES: Clc1ncn(-c2csc(N3CCOCC3)n2)c1Br] | 4-[4-(5-bromo-4-chloro- imidazol-1-yl)thiazol-2- yl]morpholine | 330 | |
| 45 | 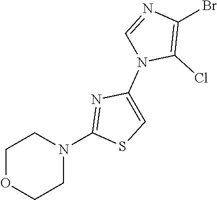 [SMILES: Clc1c(Br)ncn1-c1csc(N2CCOCC2)n1] | 4-[4-(4-bromo-5-chloro- imidazol-1-yl)thiazol-2- yl]morpholine | 1300 | |
| 46 | 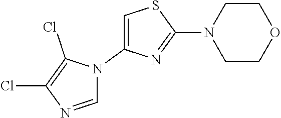 [SMILES: Clc1ncn(-c2csc(N3CCOCC3)n2)c1Cl] | 4-[4-(4,5-dichloroimidazol-1- yl)thiazol-2-yl]morpholine | 660 | |
| 47 | 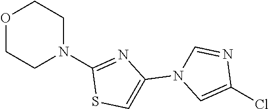 [SMILES: Clc1cn(-c2csc(N3CCOCC3)n2)cn1] | 4-[4-(4-chloroimidazol-1- yl)thiazol-2-yl]morpholine | 970 | |
| 48 | 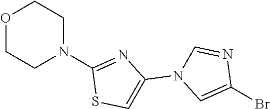 [SMILES: Brc1cn(-c2csc(N3CCOCC3)n2)cn1] | 4-[4-(4-bromoimidazol-1- yl)thiazol-2-yl]morpholine | 2100 | |
| 49 | 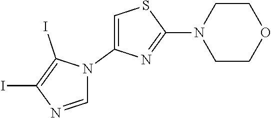 [SMILES: Ic1ncn(-c2csc(N3CCOCC3)n2)c1I] | 4-[4-(4,5-diiodoimidazol-1- yl)thiazol-2-yl]morpholine | 1500 | |
| 50 | 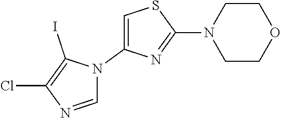 [SMILES: Clc1ncn(-c2csc(N3CCOCC3)n2)c1I] | 4-[4-(4-chloro-5-iodo- imidazol-1-yl)thiazol-2- yl]morpholine | 2440 | |
| 51 | 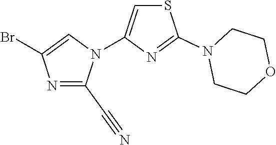 [SMILES: N#Cc1nc(Br)cn1-c1csc(N2CCOCC2)n1] | 4-bromo-1-(2- morpholinothiazol-4- yl)imidazole-2-carbonitrile | 1600 | |
| 52 | 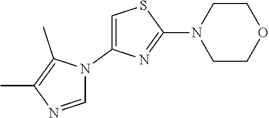 [SMILES: Cc1ncn(-c2csc(N3CCOCC3)n2)c1C] | 4-[4-(4,5-dimethylimidazol-1- yl)thiazol-2-yl]morpholine | 3650 | |
| 53 | 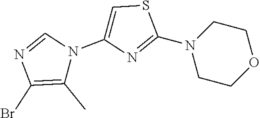 [SMILES: Cc1c(Br)ncn1-c1csc(N2CCOCC2)n1] | 4-[4-(4-bromo-5-methyl- imidazol-1-yl)thiazol-2- yl]morpholine | 44% inhib. at 1 μM | |
| 54 | 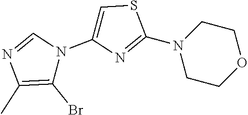 [SMILES: Cc1ncn(-c2csc(N3CCOCC3)n2)c1Br] | 4-[4-(5-bromo-4-methyl- imidazol-1-yl)thiazol-2- yl]morpholine | 28% inhib. at 1 μM | |
| 55 | 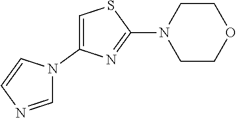 [SMILES: c1cn(-c2csc(N3CCOCC3)n2)cn1] | 4-(4-imidazol-1-ylthiazol-2- yl)morpholine | 17% inhib. at 1 μM | |
| 56 | 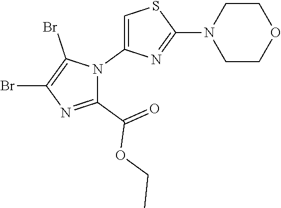 [SMILES: CCOC(=O)c1nc(Br)c(Br)n1-c1csc(N2CCOCC2)n1] | ethyl 4,5-dibromo-1-(2- morpholinothiazol-4- yl)imidazole-2-carboxylate | 12% inhib. at 1 μM | |
| 57 | 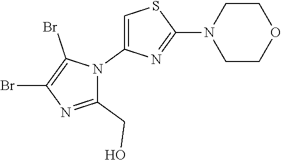 [SMILES: OCc1nc(Br)c(Br)n1-c1csc(N2CCOCC2)n1] | [4,5-dibromo-1-(2- morpholinothiazol-4- yl)imidazol-2-yl]methanol | 6450 | |
| 58 | 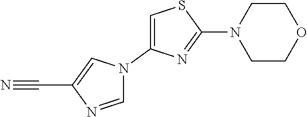 [SMILES: N#Cc1cn(-c2csc(N3CCOCC3)n2)cn1] | 1-(2-morpholinothiazol-4- yl)imidazole-4-carbonitrile | 2% inhib. at 1 μM | |
| 59 | 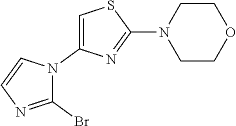 [SMILES: Brc1nccn1-c1csc(N2CCOCC2)n1] | 4-[4-(2-bromoimidazol-1- yl)thiazol-2-yl]morpholine | 35% inhib. at 1 μM | |
| 60 | 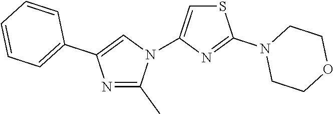 [SMILES: Cc1nc(-c2ccccc2)cn1-c1csc(N2CCOCC2)n1] | 4-[4-(2-methyl-4-phenyl- imidazol-1-yl)thiazol-2- yl]morpholine | 3% inhib. at 1 μM | |
| 61 | 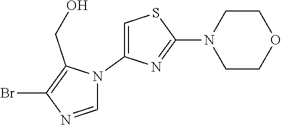 [SMILES: OCc1c(Br)ncn1-c1csc(N2CCOCC2)n1] | [5-bromo-3-(2- morpholinothiazol-4- yl)imidazol-4-yl]methanol | 31% inhib. at 1 μM | |
| 62 | 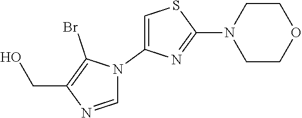 [SMILES: OCc1ncn(-c2csc(N3CCOCC3)n2)c1Br] | [5-bromo-1-(2- morpholinothiazol-4- yl)imidazol-4-yl]methanol | inact. | |
| 63 | 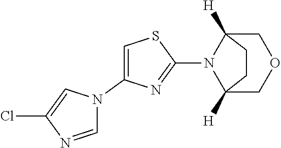 [SMILES: Clc1cn(-c2csc(N3[C@@H]4CC[C@H]3COC4)n2)cn1] | (1R,5S)-8-(4-(4-chloro-1H- imidazol-1-yl)thiazol-2-yl)-3-oxa- 8-azabicyclo[3.2.1]octane | | |
| 64 | 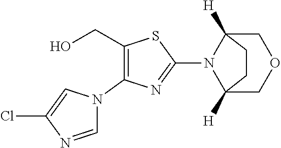 [SMILES: OCc1sc(N2[C@@H]3CC[C@H]2COC3)nc1-n1cnc(Cl)c1] | (2-((1R,5S)-3-oxa-8- azabicyclo[3.2.1]octan-8-yl)-4-(4- chloro-1H-imidazol-1-yl)thiazol-5- yl)methanol | | |
| 65 | 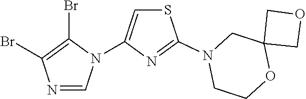 [SMILES: Brc1ncn(-c2csc(N3CCOC4(COC4)C3)n2)c1Br] | 8-(4-(4,5-dibromo-1H-imidazol-1- yl)thiazol-2-yl)-2,5-dioxa-8- azaspiro[3.5]nonane | 4471 | |
| 66 | 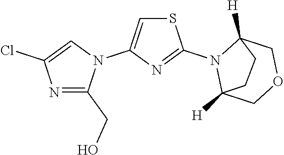 [SMILES: OCc1nc(Cl)cn1-c1csc(N2[C@@H]3CC[C@H]2COC3)n1] | (1-(2-((1R,5S)-3-oxa-8- azabicyclo[3.2.1]octan-8- yl)thiazol-4-yl)-4-chloro-1H- imidazol-2-yl)methanol | | |
| 67 | 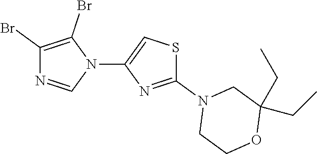 [SMILES: CCC1(CC)CN(c2nc(-n3cnc(Br)c3Br)cs2)CCO1] | 4-(4-(4,5-dibromo-1H-imidazol-1- yl)thiazol-2-yl)-2,2- diethylmorpholine | | |

For the other compounds, a single dose screen for most of them was done, while the IC₅₀ was only measured for those with a high percentage of inhibition, except a few interesting ones with relatively low % inhibition. As the % inhibition was measured at a concentration of 3 μM or 1 μM, the threshold by % inhibition for actives and inactives was low. It was found that an inhibition of about 30% at 3 μM may give IC₅₀ around 50 μM. Accordingly, generally a compound having an inhibition value lower than 30% may be considered inactive. Nevertheless, activity of a compound should not always be determined by % inhibition. In fact any compounds that were tested and considered as inactive may have been considered active if the sensitivity of the system were somewhat reduced. As compounds became better and better the level of sensitivity was increased. Accordingly, it became more difficult to measure low activity compounds in the assay system. However, since the newer compounds are based on previously successful compounds they may have some activity at some level. Accordingly, “inactive” may be an incorrect term, and “none detected” may be more appropriate. Those skilled in the art will appreciate that the point of covalent attachment of the moiety to the compounds as described herein may be, for example, and without limitation, cleaved under specified conditions. Specified conditions may include, for example, and without limitation, in vivo enzymatic or non-enzymatic means. Cleavage of the moiety may occur, for example, and without limitation, spontaneously, or it may be catalyzed, induced by another agent, or a change in a physical parameter or environmental parameter, for example, an enzyme, light, acid, temperature or pH. The moiety may be, for example, and without limitation, a protecting group that acts to mask a functional group, a group that acts as a substrate for one or more active or passive transport mechanisms, or a group that acts to impart or enhance a property of the compound, for example, solubility, bioavailability or localization.

Human microsomal stability data for two reference compounds (Imipramine and Propranolol) and representative compounds of the invention are provided in the table below:

TABLE 2
Human microsomal stability data

| | Clᵢₙₜ (μl/min/mg) |
|---|---|
| Example | human |
| Imipramine | 32 |
| Propranolol | 25 |
| 15 | 51 |
| 17 | 41 |
| 18 | 100 |
| 19 | 42 |
| 20 | 40 |
| 21 | 6 |
| 22 | 52 |
| 23 | 22 |
| 24 | 13 |
| 36 | 10 |
| 37 | 129 |
| 38 | 44 |
| 39 | 18 |
| 40 | 177 |
| 41 | 198 |
| 42 | 72 |
| 43 | 165 |
| 44 | 196 |
| 45 | 185 |
| 48 | 81 |
| 53 | 72 |
| 54 | 224 |
| 55 | 10 |
| 56 | 33 |
| 57 | 10 |
| 58 | 23 |
| 59 | 42 |
| 60 | 44 |
| 61 | 15 |
| 62 | 19 |

Representative compounds 21, 24, 36, 55, 57 and 61 demonstrate high metabolic stability in human hepatic microsomal test system. The remaining compounds tested showed moderate stability. “No cofactor” control data indicates that the low stability of compound 18 is not determined just by CYP450 activity, while for the rest of the test articles the observed rate of metabolism appears to be primarily cytochrome P450-driven.

### Materials and Methods

In Vitro Identification of the Compounds

1. Cell Culture: LNCaP and PC3 human prostate cancer cells were obtained from American Type Culture Collection (ATCC, Manassas, Va.) and grown in RPMI 1640 medium supplemented with 5% fetal bovine serum (FBS) (Invitrogen). The LNCaP eGFP cell line was stably transfected with an androgen-responsive probasin-derived promoter fused to an eGFP reporter (LN-ARR2PB-eGFP) using a lentiviral approach, and were grown in phenol-red-free RPMI 1640 supplemented with 5% CSS. In-house developed MDV3100-resistant LNCaP cells were cultured in RPMI 1640 supplemented with 5% FBS and 10 μM MDV3100. All cells were maintained at 37° C. in 5% CO₂.

2. eGFP cellular AR transcription assay: The AR transcriptional activity was assayed as previously described (Tavassoli, P., et al.¹²).

3. Prostate-specific antigen (PSA) assay: The evaluation of PSA levels secreted into the media was performed in parallel to the eGFP assay using the same plates. After cells were incubated for 3 days, 150 μl of the media was taken from each well, and added to 150 μl of PBS. PSA levels were then evaluated using Cobas e 411 analyzer instrument (Roche Diagnostics) according to the manufacturer's instructions.

4. Biolayer interferometry (BLI) assay: The direct reversible interaction between small molecules and the AR was measured as previously described (Lack, N. et al.¹³).

5. Androgen displacement assay: The androgen displacement was assessed with the Polar Screen Androgen Receptor Competitor Green Assay Kit as per the instructions of the manufacturer (Lack, N.,¹³).

6. Cell viability assay: The PC3, LNCaP, and MDV3100-resistant cells were plated at 3,000 cells per well in RPMI 1640 containing 5% charcoal stripped serum (CSS) in a 96-well plate, treated with 0.1 nM R1881 and compounds (0-25 μM) for 96 hrs. After 4 days of treatment, cell density was measured using the 3-(4, 5-dimethylthiazol-2-yl)-5-(3-carboxymethoxyphenyl)-2-(4-sulfophenyl)-2H-tetrazolium assay according to the manufacturer's protocol (CellTiter 961 Presto Blue™, ThermoFischer™).

7. Mutation Studies: The residues in the predicted binding site were mutated using the Quickchange™ Site-Directed Mutagenesis Kit as per the instructions.

8. Transient Transfection: The PC3 cells were seeded into a 96-well plate (2,000 cells/well). After 24 hrs, the wild-type AR (50 ng/well)/AR mutants and AR3TK-luciferase plasmids (1:3) were co-transfected into PC3 cells using transfection reagent TT20. 24 hrs after the transfection, the cells were treated with the compounds at various concentrations. And 24 hrs later, the cells were lysed and the reading was taken using a luminometer.

Constructs

Full-length human AR (hARWT) or splice variant (AR-V7) was encoded on a pcDNA3.1 expression plasmid. Point mutations in the DBD were generated with the QuikChange™ mutagenesis kit (Stratagene™) using hARWT or AR-V7 templates. Mutagenic primers were generated using a primer design tool (Agilent™). The glucocorticoid receptor (GR) was expressed from the pGR mammalian expression vector as described previously (Miesfeld, R. et al.¹⁴). Progesterone receptor (PR) was expressed from the pSG5-PRB vector and was obtained from Dr. X. Dong. The AR-DBD_hinge domain (amino acids 558-689) was amplified from the hARWT construct and cloned into the pTrc expression vector (Nterminal His6 tag, Invitrogen™) using the polymerase incomplete primer extension method (PIPE). Briefly, the AR-DBD+hinge domain was cloned by mixing the PCR products resulting from the following templates and primers: AR(558-689) insert from hARWT template 5′CAT CAT CAT CAT CAT CAT GGT ACC TGC CTG ATC TGT GG and 5′-CAG GCT GAA AAT CTT CTC TCA GTG TCC AGC ACA CAC TAC AC; pTrc vector lacking multiple cloning site 5′-ATCTCCACAGATCAGGCAGGT ACC ATG ATG ATG ATG ATG ATG and 5′-GGT GTA GTG TGT GCT GGA CAC-TGA GAG AAG ATT TTC AGC CTG; the underlined primer sections anneal to the specified template, although their 5′-extensions (italicized) are complementary to the corresponding primer sequence of the other PCR. Plasmid assembly is achieved by mixing the PCR products from each reaction (Klock, H. E. et al.^{5s}), followed by transformation into chemically competent bacteria. A similar strategy was used to clone the AR-DBD+hinge into the Pan4 vector (avidity) expressing the N-terminal biotinylation sequence (GLNDIFEAQKIEWHE) and C-terminal His tag. YFP-AR plasmid was a gift from Dr. Jan Trapman (van Royen, M. E. et al.¹⁶) and is based on pEYFP-C1 (Clontech™). YFP-V7 was constructed by polymerase incomplete primer extension method using the following primers and templates: AR-V7 insert from pcDNA3.1 AR-V7 template 5′-GGT GCT GGA GCA GGT GCT GGA ATG GAA GTG CAG TTA GGG CTG and 5′-GGA AAT AGG GTT TCC AAT GCT TCA GGG TCT GGT CAT TTT GAG; pEYFPC1 vector lacking the full-length AR 5′-CAG CCC TAA CTG CAC TTC CAT TCC AGC ACC TGC TCC AG and 5′-CTC AAA ATG ACC AGA CCC TGA AGC ATT GGA AAC CCT ATT TCC.

Cell Culture, Transfection, and Luciferase Assays

PC3 human PCa cells (AATC) were serum-starved in RPMI 1640 media (Invitrogen™) supplemented with 5% charcoal-stripped serum (CSS) (RPMI 1640 medium with 5% CSS) for 5 days prior to transfection. For luciferase assays, PC3 cells were seeded in 96-well plates (5000 cells/well) in RPMI 1640 medium with 5% CSS for 24 h, followed by transfection with 50 ng of hAR or other nuclear receptor plasmid, 50 ng of ARR3tk-luciferase, and 0.3 μl/well TransIT20/20 transfection reagent (TT20, Mirus™) for 48 h. Cells were then treated with compounds at various concentrations and 0.1 nMR1881 (in 100% ethanol) for 24 h. GR or PR activation was stimulated with 1 nM dexamethasone or progesterone, respectively. ER-α transcriptional activity was measured with a MCF-7 cell line bearing the stable transfection of an estrogen-response element-luciferase reporter, with transcriptional activity stimulated by 1 nM estradiol. Cell lysis was carried out with 60 μl of 1× passive lysis buffer/well (Promega™). 20 μl of cell lysate from each well were mixed with 50 μl of luciferase assay reagent (Promega™), and luminescence was recorded on a TECAN™ M200pro plate reader. Luciferase assays with splice variant AR were performed the same way but with only 5 ng of pcDNA3.1 AR-V7 (to limit the high level of AR-V7 expression) and no R1881. R1-AD 1 and TALEN-engineered R1-D567 cell lines have been described previously (Nyquist, M. D. et al.¹⁷). Assays with R1-AD1 and R1-D567 cells were performed as above but with transfection of only ARR3tk-luciferase reporter and 10,000 cells/well.

Western Blots

Cell lysates (40 μl) from luciferase assays (96-well plate) were separated on a 10% SDSpolyacrylamide mini gel. Protein was transferred to methanol-charged PVDF membranes and probed with anti-AR441 (mouse, Sigma™) monoclonal primary antibody. Blots were also probed with polyclonal anti-actin (rabbit, Sigma™) to show equal loading and polyclonal anti-PARP/anti-cleaved PARP (rabbit, Sigma™) to test for induction of apoptosis. Lysates from CWR-R1 cells were additionally probed with polyclonal anti-FKBP5 (rabbit, Sigma™) following 2 days of incubation with compounds.

Microsomal Stability Assays

Microsomal incubations were carried out in 96-well plates in 5 aliquots of 40 μL each (one for each time point). Liver microsomal incubation medium contained PBS (100 mM, pH 7.4), MgCl2 (3.3 mM), NADPH (3 mM), glucose-6-phosphate (5.3 mM), glucose-6-phosphate dehydrogenase (0.67 units/ml) and 0.41 mg of liver microsomal protein per ml. Control incubations were performed replacing the NADPH-cofactor system with PBS. Test compound (2 μM, final solvent concentration 1.6%) was incubated with microsomes at 37° C., shaking at 100 rpm. Incubations were performed in duplicates. Five time points over 40 minutes had been analyzed. The reactions were stopped by adding 12 volumes of 90% acetonitrile-water to incubation aliquots, followed by protein sedimentation by centrifuging at 5500 rpm for 5 minutes. Supernatants were analyzed using the HPLC system coupled with tandem mass spectrometer. The elimination constant (kel), half-life (t1/2) and intrinsic clearance (Clint) were determined in plot of ln(AUC) versus time, using linear regression analysis:

$${k}_{el} = - slope$$

$${t}_{1 \text{/} 2} = \frac{0.693}{k}$$

$${Cl}_{int} = \frac{0.693}{{t}_{1 \text{/} 2}} \times \frac{μ {l}_{incubation}}{{mg}_{microsomes}}$$

Reagents and Consumables

DMSO Chromasolv Plus, HPLC grade, ≥99.7% (Sigma-Aldrich™, USA; Lot #34869); Acetonitrile Chromasolv, gradient grade, for HPLC, ≥99.9% (Sigma-Aldrich™, USA; Lot #34851); Potassium phosphate monobasic ACS Grade (Helicon™, Lot # Am0781); Potassium phosphate dibasic ACS Grade (Helicon™, Lot # Am-0705); Magnesium chloride hexahydrate (Helicon™, Lot # Am-0288); Human liver microsomes, InVitroCYP Mclass (BioreclamationlVT™, Product # X008069, Lot HPH); Glucose-6-phosphate dehydrogenase from baker's yeast (S. cerevisiae), type XV (Sigma-Aldrich™, USA; Lot # G6378); Glucose-6-phosphate sodium salt, Sigma Grade™, crystalline (Sigma-Aldrich™, USA; Lot # G7879); NADPH tetrasodium salt, ≥95% (Santa Cruz Biotechnology, Inc.™, USA; Lot # sc-202725); Formic acid for mass spectrometry, ˜98% (Fluka™, USA; Lot #94318); DMSO stock solution of the test compounds 10 mM; Propranolol hydrochloride ≥99% (TLC), powder (Sigma-Aldrich™, USA; Lot # P0884); Imipramine hydrochloride ≥99% (TLC) (Sigma-Aldrich™, USA; Lot #17379); Zorbax Eclipse Plus C18 column 2.1×50 mm, 3.5 m (Agilent Technologies, Inc.™ USA); and 1.1 ml microtubes in microracks, pipettor tips (Thermo Scientific™, USA).

Equipment

Gradient HPLC system VP (Shimadzu™, Japan); MS/MS detector API 3000 PE with TurbolonSpray Electrospray™ module (PE Sciex™, USA); VWR Membrane Nitrogen Generators N2-04-L1466, nitrogen purity 99%+(VWR™, USA); Innova 4080 Incubator Shaker™ (New Brunswick Scientific™, USA); Water purification system NANOpure Diamond D11911™ (Thermo Scientific™, Barnstead, USA); and Multichannel Electronic Pipettes 2-125 μL, 5-250 μL, 15-1250 μL, Matrix (Thermo Scientific™, USA; Cat ##2001, 2002, 2004). Analytical System All measurements were performed using Shimadzu VP HPLC system including vacuum degasser, gradient pumps, reverse phase HPLC column, column oven and autosampler. The HPLC system was coupled with tandem mass spectrometer API 3000™ (PE Sciex™). The TurbolonSpray ion source was used in both positive and negative ion modes. Acquisition and analysis of the data were performed using Analyst 1.5.2™ software (PE Sciex™).

Additional reference: Dalal K. et al.¹⁸

### Pharmaceutical Compositions

The compounds of formula I and the pharmaceutically acceptable salts can be used as therapeutically active substances, e.g. in the form of pharmaceutical preparations. The pharmaceutical preparations can be administered orally, e.g. in the form of tablets, coated tablets, dragées, hard and soft gelatin capsules, solutions, emulsions or suspensions. The administration can, however, also be effected rectally, e.g. in the form of suppositories, or parenterally, e.g. in the form of injection solutions.

The compounds of formula I and the pharmaceutically acceptable salts thereof can be processed with pharmaceutically inert, inorganic or organic carriers for the production of pharmaceutical preparations. Lactose, corn starch or derivatives thereof, talc, stearic acids or its salts and the like can be used, for example, as such carriers for tablets, coated tablets, dragées and hard gelatin capsules. Suitable carriers for soft gelatin capsules are, for example, vegetable oils, waxes, fats, semi-solid and liquid polyols and the like. Depending on the nature of the active substance no carriers are however usually required in the case of soft gelatin capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical preparations can, moreover, contain pharmaceutically acceptable auxiliary substances such as preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Medicaments containing a compound of formula I or a pharmaceutically acceptable salt thereof and a therapeutically inert carrier are also provided by the present invention, as is a process for their production, which comprises bringing one or more compounds of formula I and/or pharmaceutically acceptable salts thereof and, if desired, one or more other therapeutically valuable substances into a galenical administration form together with one or more therapeutically inert carriers.

The dosage can vary within wide limits and will, of course, have to be adjusted to the individual requirements in each particular case. In the case of oral administration the dosage for adults can vary from about 0.01 mg to about 1000 mg per day of a compound of general formula I or of the corresponding amount of a pharmaceutically acceptable salt thereof. The daily dosage may be administered as single dose or in divided doses and, in addition, the upper limit can also be exceeded when this is found to be indicated.

The following examples illustrate the present invention without limiting it, but serve merely as representative thereof. The pharmaceutical preparations conveniently contain about 1-500 mg, particularly 1-100 mg, of a compound of formula I. Examples of compositions according to the invention are:

### Example A

Tablets of the following composition are manufactured in the usual manner:

TABLE 3
possible tablet composition

mg/tablet

| ingredient | 5 | 25 | 100 | 500 |
|---|---|---|---|---|
| Compound of formula I | 5 | 25 | 100 | 500 |
| Lactose Anhydrous DTG | 125 | 105 | 30 | 150 |
| Sta-Rx 1500 | 6 | 6 | 6 | 60 |
| Microcrystalline Cellulose | 30 | 30 | 30 | 450 |
| Magnesium Stearate | 1 | 1 | 1 | 1 |
| Total | 167 | 167 | 167 | 831 |

### Manufacturing Procedure

1. Mix ingredients 1, 2, 3 and 4 and granulate with purified water.
2. Dry the granules at 50° C.
3. Pass the granules through suitable milling equipment.
4. Add ingredient 5 and mix for three minutes; compress on a suitable press.

### Example B-1

Capsules of the following composition are manufactured:

TABLE 4
possible capsule ingredient composition

mg/capsule

| ingredient | 5 | 25 | 100 | 500 |
|---|---|---|---|---|
| Compound of formula I | 5 | 25 | 100 | 500 |
| Hydrous Lactose | 159 | 123 | 148 | — |
| Corn Starch | 25 | 35 | 40 | 70 |
| Talk | 10 | 15 | 10 | 25 |
| Magnesium Stearate | 1 | 2 | 2 | 5 |
| Total | 200 | 200 | 300 | 600 |

### Manufacturing Procedure

1. Mix ingredients 1, 2 and 3 in a suitable mixer for 30 minutes.
2. Add ingredients 4 and 5 and mix for 3 minutes.
3. Fill into a suitable capsule.

The compound of formula I, lactose and corn starch are firstly mixed in a mixer and then in a comminuting machine. The mixture is returned to the mixer; the talc is added thereto and mixed thoroughly. The mixture is filled by machine into suitable capsules, e.g. hard gelatin capsules.

### Example B-2

Soft Gelatin Capsules of the following composition are manufactured:

TABLE 5
possible soft gelatin capsule ingredient composition

| ingredient | mg/capsule |
|---|---|
| Compound of formula I | 5 |
| Yellow wax | 8 |
| Hydrogenated Soya bean oil | 8 |
| Partially hydrogenated plant oils | 34 |
| Soya bean oil | 110 |
| Total | 165 |

TABLE 6
possible soft gelatin capsule composition

| ingredient | mg/capsule |
|---|---|
| Gelatin | 75 |
| Glycerol 85% | 32 |
| Karion 83 | 8 (dry matter) |
| Titan dioxide | 0.4 |
| Iron oxide yellow | 1.1 |
| Total | 116.5 |

### Manufacturing Procedure

The compound of formula I is dissolved in a warm melting of the other ingredients and the mixture is filled into soft gelatin capsules of appropriate size. The filled soft gelatin capsules are treated according to the usual procedures.

### Example C

Suppositories of the following composition are manufactured:

TABLE 7
possible suppository composition

| ingredient | mg/supp. |
|---|---|
| Compound of formula I | 15 |
| Suppository mass | 1285 |
| Total | 1300 |

### Manufacturing Procedure

The suppository mass is melted in a glass or steel vessel, mixed thoroughly and cooled to 45° C. Thereupon, the finely powdered compound of formula I is added thereto and stirred until it has dispersed completely. The mixture is poured into suppository moulds of suitable size, left to cool; the suppositories are then removed from the moulds and packed individually in wax paper or metal foil.

### Example D

Injection solutions of the following composition are manufactured:

TABLE 8
possible injection solution composition

| ingredient | mg/injection solution. |
|---|---|
| Compound of formula I | 3 |
| Polyethylene Glycol 400 | 150 |
| acetic acid | q.s. ad pH 5.0 |
| water for injection solutions | ad 1.0 ml |

### Manufacturing Procedure

The compound of formula I is dissolved in a mixture of Polyethylene Glycol 400 and water for injection (part). The pH is adjusted to 5.0 by acetic acid. The volume is adjusted to 1.0 ml by addition of the residual amount of water. The solution is filtered, filled into vials using an appropriate overage and sterilized.

### Example E

Sachets of the following composition are manufactured:

TABLE 9
possible sachet composition

| ingredient | mg/sachet |
|---|---|
| Compound of formula I | 50 |
| Lactose, fine powder | 1015 |
| Microcrystalline cellulose (AVICEL PH 102) | 1400 |
| Sodium carboxymethyl cellulose | 14 |
| Polyvinylpyrrolidon K 30 | 10 |
| Magnesium stearate | 10 |
| Flavoring additives | 1 |
| Total | 2500 |

### Manufacturing Procedure

The compound of formula I is mixed with lactose, microcrystalline cellulose and sodium carboxymethyl cellulose and granulated with a mixture of polyvinylpyrrolidone in water. The granulate is mixed with magnesium stearate and the flavoring additives and filled into sachets.

### Experimental Part

The following examples are provided for illustration of the invention. They should not be considered as limiting the scope of the invention, but merely as being representative thereof.

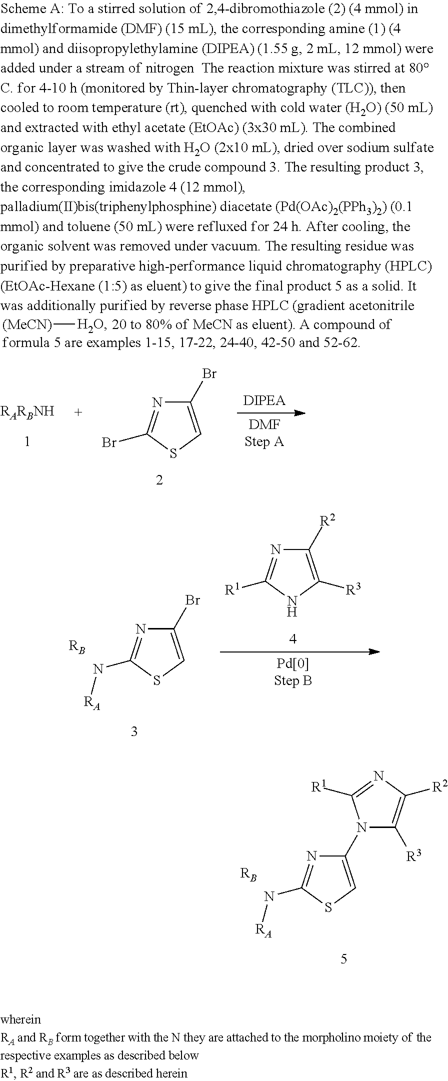

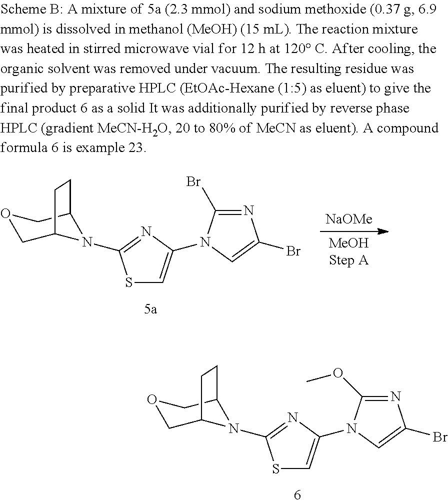 [SMILES: Brc1cn(-c2csc(N3C4CCC3COC4)n2)c(Br)n1 ; COc1nc(Br)cn1-c1csc(N2C3CCC2COC3)n1 ; "NaOMe"]

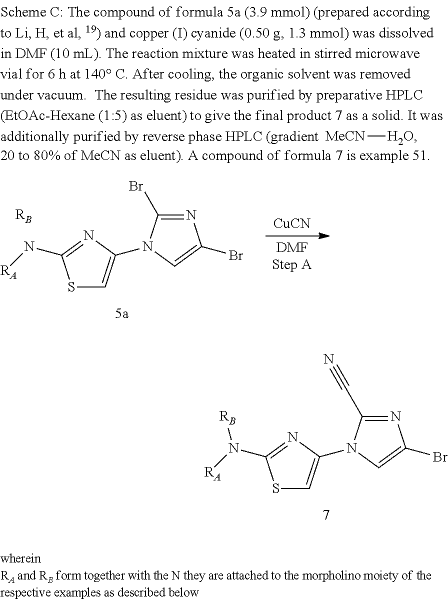

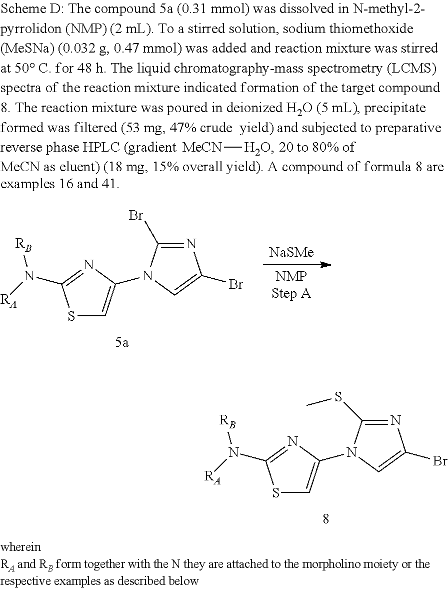

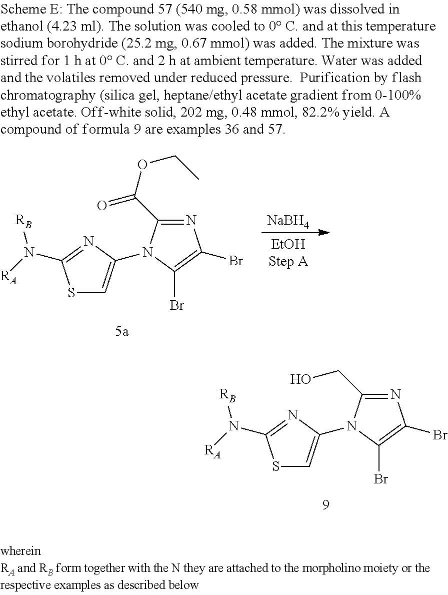

¹ W. C. Krause et al., The International Journal of Biochemistry & Cell Biology, Vol 54, September 2014, p 49-59
² Scher et al., JAMA Oncol., doi:10.1001/jamaoncol.2016.1828, Jun. 4, 2016
³ Damber et al., Lancet, May 17, 2008, 371(9625), p 1710-21
⁴ Hickey et al., Oncotarge Dec. 29, 2015, 6(42), 6296
⁵ E. Antonarakis, Special Issue of Cancers (ISSN 2072-6694) “AR Signaling in Human Malignancies: Prostate Cancer and Beyond”, http://www.mdpi.com/joumal/cancers/special_issues/ar_signal, 2016
⁶ Luo J., Asian J Androl, 2016, 18(4), 580-5
⁷ WO2015120543
⁸ WO2015065919
⁹ Li et al. J. Med. Chem., 2014, 57 (15), pp 6458-6467
¹⁰ Dalal et al., J Biol Chem. 2014 Sep. 19; 289(38): 26417-26429.
¹¹ Irwin, J. et al. Abstracts of Papers Am. Chem. Soc. (2005) 230:U1009
¹² Tavassoli, P., et al. (2007). Prostate 67(4): 416-426
¹³ Lack, N. et al. (2011). Journal of Medicinal Chemistry 54(24):8563-73
¹⁴ Miesfeld, R. et al. (1986) Genetic complementation of a glucocorticoid receptor deficiency by expression of cloned receptor cDNA. Cell 46, 389-399
¹⁵ Klock, H. E. et al. (2008) combining the polymerase incomplete primer extension method for cloning and mutagenesis with microscreening to accelerate structural genomics efforts. Proteins 71, 982-994
¹⁶ van Royen, M. E. et al. (2012) Stepwise androgen receptor dimerization. J. Cell Sci. 125, 1970-1979
¹⁷ Nyquist, M. D. et al. (2013) TALEN-engineered AR gene rearrangements reveal endocrine uncoupling of androgen receptor in prostate cancer. Proc. Natl. Acad. Sci. U.S.A. 110, 17492-17497
¹⁸ Dalal K. et al. “Selectively Targeting the DNA-binding Domain of the Androgen Receptor as a Prospective Therapy for Prostate Cancer” THE JOURNAL OF BIOLOGICAL CHEMISTRY VOL. 289, NO. 38, pp. 26417-26429, Sep. 19, 2014 (published online 1 Aug. 2014)
¹⁹ Li, H. et al. J. Med. Chem. 2014, 57, 6458-6467; DOI: 10.1021/jm500802j

1. A compound of formula I, 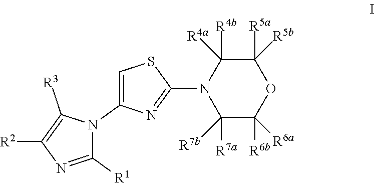
wherein
R¹ is selected from the group consisting of
i) hydrogen,
ii) halogen,
iii) CN,
iv) —C(═O)O—C₁₋₆-alkyl,
v) hydroxy-C₁₋₆-alkyl, and
vi) —X₀₋₁—C₁₋₆-alkyl;
R² is selected from the group consisting of
i) aryl,
ii) hydrogen,
iii) halogen,
iv) CN,
v) hydroxy-C₁₋₆-alkyl, and
vi) C₁₋₆-alkyl;
R³ is selected from the group consisting of
i) hydrogen,
ii) halogen,
iii) hydroxy-C₁₋₆-alkyl, and
iv) C₁₋₆-alkyl;
R^{4a} is selected from the group consisting of
i) hydrogen,
ii) C₃₋₇-cycloalkyl, and
iii) C₁₋₆-alkyl;
R^{4b} is hydrogen or —(CH₂)₁₋₄— together with
i) R^{5b},
ii) R^{6b}, or
iii) R^{7b};
R^{5a} is selected from the group consisting of
i) aryl,
ii) heteroaryl,
iii) hydrogen,
iv) halogen-C₁₋₆-alkyl, and
v) C₁₋₆-alkyl; or
vi) together with R^{5b} is
a. —(CH₂)₁₋₄—, or
b. —(CH₂)₁₋₂—O—(CH₂)₁₋₂—;
R^{5b} is selected from the group consisting of
i) hydrogen, and
ii) C₁₋₆-alkyl; or
iii) together with R^{5a} is
a. —(CH₂)₁₋₄—, or
b. —(CH₂)₁₋₂—O—(CH₂)₁₋₂—; or
iv) together with R^{4b} is —(CH₂)₁₋₄—, or
v) together with R^{6b} is —(CH₂)₁₋₄—, or,
vi) together with R^{7b} is —(CH₂)₁₋₄—;
R^{6a} is selected from the group consisting of
i) hydrogen, and
ii) C₁₋₆-alkyl;
R^{6b} is selected from the group consisting of
i) hydrogen, and
ii) C₁₋₆-alkyl; or
iii) —(CH₂)₁₋₄— together with
a. R^{4b}, or
b. R^{5b};
R^{7a} is hydrogen;
R^{7b} is selected from the group consisting of
i) hydrogen, and
ii) —(CH₂)₁₋₄— together with R^{4b};
X is selected from the group consisting of
i) S, and
ii) O;
or pharmaceutically acceptable salts thereof.

2. The compound of formula I according to claim 1, wherein
R¹ is selected from the group consisting of
i) hydrogen,
ii) Br,
iii) CN,
iv) —C(═O)O—CH₂CH₃,
v) hydroxy-CH₂—, and
vi) —S—CH₃,
vii) —O—CH₃, and
viii) CH₃.

3. The compound of formula I according to claim 1, wherein R¹ is Br.

4. The compound of formula I according to claim 1, wherein
R² is selected from the group consisting of
i) phenyl,
ii) hydrogen,
iii) Br,
iv) Cl,
v) I,
vi) CN,
vii) hydroxy-CH₂—, and
viii) CH₃.

5. The compound of formula I according to claim 1, wherein R² is Br.

6. The compound of formula I according to claim 1, wherein
R³ is selected from the group consisting of
i) hydrogen,
ii) Br,
iii) Cl,
iv) I,
v) hydroxy-CH₂—, and
vi) CH₃.

7. The compound of formula I according to claim 1, wherein R³ is hydrogen.

8. The compound of formula I according to claim 1, wherein the morpholino moiety is bridged by a —(CH₂)₁₋₃— moiety.

9. The compound of formula I according to claim 1, selected from the group consisting of:
(1-(2-((1R,5S)-3-oxa-8-azabicyclo[3.2.1]octan-8-yl)thiazol-4-yl)-4-chloro-1H-imidazol-2-yl)methanol,
(1R,5S)-3-[4-(4,5-dibromoimidazol-1-yl)thiazol-2-yl]-8-oxa-3-azabicyclo[3.2.1]octane,
(1R,5S)-3-[4-(4-bromo-5-chloro-imidazol-1-yl)thiazol-2-yl]-8-oxa-3-azabicyclo[3.2.1]octane,
(1R,5S)-3-[4-(5-bromo-4-chloro-imidazol-1-yl)thiazol-2-yl]-8-oxa-3-azabicyclo[3.2.1]octane,
(1R,5S)-8-(4-(4-chloro-1H-imidazol-1-yl)thiazol-2-yl)-3-oxa-8-azabicyclo[3.2.1]octane,
(1R,5S)-8-[4-(2,4-dibromoimidazol-1-yl)thiazol-2-yl]-3-oxa-8-azabicyclo[3.2.1]octane,
(1R,5S)-8-[4-(4,5-dibromoimidazol-1-yl)thiazol-2-yl]-3-oxa-8-azabicyclo[3.2.1]octane,
(1R,5S)-8-[4-(4,5-dichloroimidazol-1-yl)thiazol-2-yl]-3-oxa-8-azabicyclo[3.2.1]octane,
(1R,5S)-8-[4-(4-bromo-2-methylsulfanyl-imidazol-1-yl)thiazol-2-yl]-3-oxa-8-azabicyclo[3.2.1]octane,
(1R,5S)-8-[4-(4-bromo-5-chloro-imidazol-1-yl)thiazol-2-yl]-3-oxa-8-azabicyclo[3.2.1]octane,
(1R,5S)-8-[4-(5-bromo-4-chloro-imidazol-1-yl)thiazol-2-yl]-3-oxa-8-azabicyclo[3.2.1]octane,
(1R,5S)-9-[4-(4-bromo-5-chloro-imidazol-1-yl)thiazol-2-yl]-3-oxa-9-azabicyclo[3.3.1]nonane,
(1R,5S)-9-[4-(5-bromo-4-chloro-imidazol-1-yl)thiazol-2-yl]-3-oxa-9-azabicyclo[3.3.1]nonane,
(1S,4S)-5-[4-(2,4-dibromoimidazol-1-yl)thiazol-2-yl]-2-oxa-5-azabicyclo[2.2.1]heptane,
(1S,4S)-5-[4-(4,5-dibromoimidazol-1-yl)thiazol-2-yl]-2-oxa-5-azabicyclo[2.2.1]heptane,
(1S,5R)-3-[4-(2,4-dibromoimidazol-1-yl)thiazol-2-yl]-8-oxa-3-azabicyclo[3.2.1]octane,
(1S,5R)-3-[4-(4,5-dichloroimidazol-1-yl)thiazol-2-yl]-8-oxa-3-azabicyclo[3.2.1]octane,
(1S,5R)-8-[4-(4-bromo-2-methoxy-imidazol-1-yl)thiazol-2-yl]-3-oxa-8-azabicyclo[3.2.1]octane,
(1S,5R)-9-[4-(2,4-dibromoimidazol-1-yl)thiazol-2-yl]-3-oxa-9-azabicyclo[3.3.1]nonane,
(1S,5R)-9-[4-(4,5-dibromoimidazol-1-yl)thiazol-2-yl]-3-oxa-9-azabicyclo[3.3.1]nonane,
(2-((1R,5S)-3-oxa-8-azabicyclo[3.2.1]octan-8-yl)-4-(4-chloro-1H-imidazol-1-yl)thiazol-5-yl)methanol,
[4,5-dibromo-1-[2-[(1R,5S)-3-oxa-8-azabicyclo[3.2.1]octan-8-yl]thiazol-4-yl]imidazol-2-yl]methanol,
[5-bromo-1-(2-morpholinothiazol-4-yl)imidazol-4-yl]methanol,
[5-bromo-3-(2-morpholinothiazol-4-yl)imidazol-4-yl]methanol,
1-(2-morpholinothiazol-4-yl)imidazole-4-carbonitrile,
2,6-ditert-butyl-4-[4-(2,4-dibromoimidazol-1-yl)thiazol-2-yl]morpholine,
2-benzyl-4-[4-(2,4-dibromoimidazol-1-yl)thiazol-2-yl]morpholine,
3-cyclopropyl-4-[4-(2,4-dibromoimidazol-1-yl)thiazol-2-yl]morpholine,
4-(4-(4,5-dibromo-1H-imidazol-1-yl)thiazol-2-yl)-2,2-diethylmorpholine,
4-(4-imidazol-1-ylthiazol-2-yl)morpholine,
4-[4-(2,4-dibromoimidazol-1-yl)thiazol-2-yl]-1,9-dioxa-4-azaspiro[5.5]undecane,
4-[4-(2,4-dibromoimidazol-1-yl)thiazol-2-yl]-2-(2-thienyl)morpholine,
4-[4-(2,4-dibromoimidazol-1-yl)thiazol-2-yl]-2-(trifluoromethyl)morpholine,
4-[4-(2,4-dibromoimidazol-1-yl)thiazol-2-yl]-2-(trifluoromethyl)morpholine,
4-[4-(2,4-dibromoimidazol-1-yl)thiazol-2-yl]-2,2-diethyl-morpholine,
4-[4-(2,4-dibromoimidazol-1-yl)thiazol-2-yl]-2,2-dimethyl-morpholine,
4-[4-(2,4-dibromoimidazol-1-yl)thiazol-2-yl]-2,3,4a,5,6,7,8,8a-octahydrobenzo[b][1,4]oxazine,
4-[4-(2,4-dibromoimidazol-1-yl)thiazol-2-yl]-2,5-dimethyl-morpholine,
4-[4-(2,4-dibromoimidazol-1-yl)thiazol-2-yl]-2-isopropyl-morpholine,
4-[4-(2,4-dibromoimidazol-1-yl)thiazol-2-yl]-2-methyl-morpholine,
4-[4-(2,4-dibromoimidazol-1-yl)thiazol-2-yl]-2-phenyl-morpholine,
4-[4-(2,4-dibromoimidazol-1-yl)thiazol-2-yl]-3,4a, 5,6,7,7a-hexahydro-2H-cyclopenta[b][1,4]oxazine,
4-[4-(2,4-dibromoimidazol-1-yl)thiazol-2-yl]-3-ethyl-morpholine,
4-[4-(2,4-dibromoimidazol-1-yl)thiazol-2-yl]morpholine,
4-[4-(2-bromoimidazol-1-yl)thiazol-2-yl]morpholine,
4-[4-(2-methyl-4-phenyl-imidazol-1-yl)thiazol-2-yl]morpholine,
4-[4-(4,5-dibromoimidazol-1-yl)thiazol-2-yl]-2,6-dimethyl-morpholine
4-[4-(2,4-dibromoimidazol-1-yl)thiazol-2-yl]-2,6-dimethyl-morpholine,
4-[4-(4,5-dibromoimidazol-1-yl)thiazol-2-yl]morpholine,
4-[4-(4,5-dichloroimidazol-1-yl)thiazol-2-yl]morpholine,
4-[4-(4,5-diiodoimidazol-1-yl)thiazol-2-yl]morpholine,
4-[4-(4,5-dimethylimidazol-1-yl)thiazol-2-yl]morpholine,
4-[4-(4-bromo-2-methyl-imidazol-1-yl)thiazol-2-yl]morpholine,
4-[4-(4-bromo-2-methylsulfanyl-imidazol-1-yl)thiazol-2-yl]morpholine,
4-[4-(4-bromo-5-chloro-imidazol-1-yl)thiazol-2-yl]morpholine,
4-[4-(4-bromo-5-methyl-imidazol-1-yl)thiazol-2-yl]morpholine,
4-[4-(4-bromoimidazol-1-yl)thiazol-2-yl]morpholine,
4-[4-(4-chloro-5-iodo-imidazol-1-yl)thiazol-2-yl]morpholine,
4-[4-(4-chloroimidazol-1-yl)thiazol-2-yl]morpholine,
4-[4-(5-bromo-4-chloro-imidazol-1-yl)thiazol-2-yl]morpholine,
4-[4-(5-bromo-4-methyl-imidazol-1-yl)thiazol-2-yl]morpholine,
4-bromo-1-(2-morpholinothiazol-4-yl)imidazole-2-carbonitrile,
5-[4-(2,4-dibromoimidazol-1-yl)thiazol-2-yl]-2-oxa-5-azabicyclo[2.2.1]heptane,
8-(4-(4,5-dibromo-1H-imidazol-1-yl)thiazol-2-yl)-2,5-dioxa-8-azaspiro[3.5]nonane,
8-[4-(2,4-dibromoimidazol-1-yl)thiazol-2-yl]-2,5-dioxa-8-azaspiro[3.5]nonane,
9-[4-(2,4-dibromoimidazol-1-yl)thiazol-2-yl]-6-oxa-9-azaspiro[4.5]decane,
ethyl 4,5-dibromo-1-(2-morpholinothiazol-4-yl)imidazole-2-carboxylate, and
ethyl 4,5-dibromo-1-(2-morpholinothiazol-4-yl)imidazole-2-carboxylate,
or a pharmaceutically acceptable salt thereof.

10. (canceled)

11. A method for the treatment of cancer, comprising the step of administering a therapeutically effective amount of a compound according to claim 1, or a pharmaceutically acceptable salt thereof, to a patient in need thereof.

12. (canceled)

13. A pharmaceutical composition, comprising a compound of formula I, or a pharmaceutically acceptable salt thereof, according to claim 1 and a pharmaceutically acceptable carrier and/or a pharmaceutically acceptable auxiliary substance.

14. A method of treating an AR-V7-positive patient suffering from cancer, comprising the steps of determining the AR-V7-status in said patient and administering the compound of formula I, or a pharmaceutically acceptable salt thereof, according to claim 1 to said patient.

15. (canceled)

16. The method according to claim 11, wherein said cancer is prostate cancer, breast cancer, bladder cancer, endometrial and ovarian cancers, hepatocellular and pancreatic cancers, and salivary gland cancers.

17. The method according to claim 14, wherein said cancer is prostate cancer.


SEQUENCE LISTING (9 sequences)

SEQ ID NO: 1 - DNA, 38 bp, Artificial sequence
  hARWT template 1 DNA
catcatcatcatcatcatggtacctgcctgatctgtgg

SEQ ID NO: 2 - DNA, 41 bp, Artificial sequence
  hARWT template 2 DNA
caggctgaaaatcttctctcagtgtccagcacacactacac

SEQ ID NO: 3 - DNA, 42 bp, Artificial sequence
  pTrc 1
atctccacagatcaggcaggtaccatgatgatgatgatgatg

SEQ ID NO: 4 - DNA, 42 bp, Artificial sequence
  pTrc 2
ggtgtagtgtgtgctggacactgagagaagattttcagcctg

SEQ ID NO: 5 - protein, 15 aa, Artificial Sequence
  modified peptide
GLNDIFEAQKIEWHE

SEQ ID NO: 6 - DNA, 42 bp, Artificial sequence
  pcDNA3.1 AR-V7 template 1 DNA
ggtgctggagcaggtgctggaatggaagtgcagttagggctg

SEQ ID NO: 7 - DNA, 42 bp, Artificial sequence
  pcDNA3.1 AR-V7 template DNA
ggaaatagggtttccaatgcttcagggtctggtcattttgag

SEQ ID NO: 8 - DNA, 38 bp, Artificial sequence
  pEYFPC1 vector 1 DNA
cagccctaactgcacttccattccagcacctgctccag

SEQ ID NO: 9 - DNA, 42 bp, Artificial sequence
  pEYFPC1 vector 2 DNA
ctcaaaatgaccagaccctgaagcattggaaaccctatttcc